SEQUENCE LISTING (13 sequences)

SEQ ID NO: 1 - protein, 12 aa, Artificial Sequence
  Synthetic peptide
PEHWSYDKLRPG

SEQ ID NO: 2 - protein, 20 aa, Artificial Sequence
  Synthetic peptide
GTRIIYDRKFLMECRNSPVT

SEQ ID NO: 3 - protein, 20 aa, Artificial Sequence
  Synthetic peptide
GTRIIYDRKFLLDRRNSPMA

SEQ ID NO: 4 - protein, 20 aa, Artificial Sequence
  Synthetic peptide
GTRIIYDRKFLLECKNSPIA

SEQ ID NO: 5 - protein, 28 aa, Artificial Sequence
  Synthetic peptide
GTRIIYDRKFLMLEDCRRKNSPVMITAA

SEQ ID NO: 6 - protein, 32 aa, Artificial Sequence
  Synthetic peptide
PEHWSYDKLRPGGTRIIYDRKFLMECRNSPVT

SEQ ID NO: 7 - protein, 32 aa, Artificial Sequence
  Synthetic peptide
PEHWSYDKLRPGGTRIIYDRKFLLDRRNSPMA

SEQ ID NO: 8 - protein, 11 aa, Artificial Sequence
  Synthetic peptide
YGRKKRRQRRR

SEQ ID NO: 9 - protein, 31 aa, Artificial Sequence
  Synthetic peptide
YGRKKRRQRRRGTRIIYDRKFLMECRNSPVT

SEQ ID NO: 10 - protein, 31 aa, Artificial Sequence
  Synthetic peptide
YGRKKRRQRRRGTRIIYDRKFLLDRRNSPMA

SEQ ID NO: 11 - protein, 23 aa, Artificial Sequence
  Synthetic peptide
GLFEAIEGFIENGWEGMIDGWYG

SEQ ID NO: 12 - protein, 70 aa, Artificial Sequence
  Synthetic peptide
MKFLVNVALVFMVVYISYIYAAPEPEPAPEPEAEADAEADPEAGIGAVLKVLTTGLPALISWIKRKRQQG

SEQ ID NO: 13 - protein, 26 aa, Artificial Sequence
  Synthetic peptide
GIGAVLKVLTTGLPALISWIKRKRQQ

(19) United States
(12) Patent Application Publication
(10) Pub. No.: US 2011/0319338 A1
(43) Pub. Date: Dec. 29, 2011

(54) PEPTIDES THAT BIND EUKARYOTIC TRANSLATION INITIATION FACTOR 4E

(75) Inventor: Honami Naora, Houston, TX (US)

(73) Assignee: The Board of Regents of the University of Texas System, Austin, TX (US)

(21) Appl. No.: 13/140,722

(22) PCT Filed: Dec. 2, 2009
(86) PCT No.: PCT/US2009/066424

§ 371 Date: Sep. 6, 2011

Related U.S. Application Data
(60) Provisional application No. 61/138,753, filed on Dec. 18, 2008.

(51) Int. Cl. A61K 38/17; C07K 7/08; C07K 14/00; A61M 35/00; C07K 14/11; A61P 35/00; A61M 5/178; C12N 5/071; C07K 7/06

(52) U.S. Cl. 514/19.4; 435/375; 530/326; 530/324; 530/327; 530/396; 514/19.3; 514/19.5; 604/187; 604/289

(57) ABSTRACT

Methods, compositions and kits for treating proliferative and non-proliferative diseases associated with abnormal protein synthesis. Chimeric peptide constructs are comprised in compositions and kits for use in the treatment of proliferative diseases, such as ovarian cancer, and for inhibiting protein synthesis in a tumor cell compared to a non-tumor cell.

The present application claims the priority benefit of U.S. provisional application No. 61/138,753, filed Dec. 18, 2008, the entire contents of which are incorporated herein by reference.

### BACKGROUND OF THE INVENTION

I. Field of the Invention

The present invention relates to methods and compositions for modulating the process of protein synthesis that is controlled by the eukaryotic translation initiation factor 4E (eIF4E). The invention also relates to chimeric peptide constructs for cell-targeted treatment of proliferative diseases and conditions associated with abnormal protein synthesis.

II. Background and Description of Related Art

The process of protein synthesis is essential for cell growth and depends on the initiation of translation of messenger RNA (mRNA). Initiation of mRNA translation is tightly regulated by the eukaryotic translation initiation factor 4E (eIF4E). Many proliferative and nonproliferative diseases develop with aberrant synthesis of cellular growth factors and pathology-promoting proteins as well as dysfunctional translation initiation machinery. Inhibiting the process of protein synthesis at the step of translation initiation can therefore be an effective strategy to abrogate the growth of cancerous or otherwise diseased cells.

Because eIF4E is essential for translation initiation and thereby for protein synthesis, eIF4E is an ideal molecular target through which to inhibit cell proliferation. As such, groups have reported the application of rapamycin to inhibit phosphorylation events that increase eIF4E activity (1), and the development of antisense oligonucleotides (2) and a small molecule inhibitor (3) designed to inhibit eIF4E expression or activity, respectively. However, because eIF4E is ubiquitously expressed and important in the function of both normal and diseased cells, the principally unmet need in the development of functional eIF4E-inhibitory agents continues to be cell-specific targeting. Non-specific inhibition of eIF4E may result in serious consequences and side-effects when not designed to impact only diseased cells. Therefore, a therapeutic agent which combines specific targeting of diseased cells with preferential inhibition of eIF4E could be powerful, specific, and safe in the treatment of proliferative and some nonproliferative diseases.

One proliferative disease for which new-generation targeted therapies are a critical need is ovarian cancer which represents the fifth leading cause of cancer death for women. The epithelial type of ovarian cancer is particularly dangerous, since 70% of patients who are diagnosed with epithelial ovarian cancer present with advanced-stage disease and are rarely treated with success by conventional platinum and taxane therapies. Treatment with these conventional therapies can involve significant medical complications, and often results in long-term health consequences. Cancer-targeted inhibitors of protein synthesis would therefore represent an especially important advance in ovarian cancer treatment as an effective therapeutic approach with fewer side-effects.

### SUMMARY OF THE INVENTION

The present invention relates to methods of treating diseases, particularly proliferative disorders such as cancer, through the targeted inhibition of protein synthesis at the step of translation initiation. The invention advantageously provides chimeric peptide constructs which are designed to specifically enter cells, bind eIF4E, and disrupt the interaction of eIF4E with eIF4G, an interaction that is required to initiate translation and thereby cause synthesis of a variety of growth factors and anti-apoptotic proteins. Preferred chimeric peptide constructs of the present invention are also designed for cell specificity, enhanced serum stability, effective cell penetration, and optimal cytoplasmic localization. Pharmaceutical compositions containing these chimeric peptide constructs as well as kits and methods of use thereof are provided for treating diseases characterized by abnormal protein synthesis, abnormal cell proliferation, abnormal cell survival, and for inhibiting protein synthesis in cancer cells and in GnRH receptor-bearing cells that exhibit abnormal protein synthesis.

In particular embodiments, the present invention provides chimeric peptide constructs comprising an eIF4E binding domain and one or more domains selected from the group consisting of, wherein the eIF4E binding domain inhibits protein synthesis. A chimeric peptide construct may feature a cell targeting domain that is either N-terminal or C-terminal with respect to its eIF4E binding domain. A preferred chimeric peptide construct may comprise, for example, SEQ ID NO:6 or SEQ ID NO:7.

In some embodiments, the cell targeting domain of a chimeric peptide construct may be a GnRH receptor binding domain, in particular, a GnRH receptor type I (GnRH-RI) binding domain, and may confer preferential binding of the construct to cells having a GnRH-RI, thus inhibiting protein synthesis in a cell-specific manner. A preferred cell targeting domain may comprise at least one amino acid in the D-isomeric form, and a cell targeting domain may comprise, for example, SEQ ID NO:1.

In certain aspects of the present invention, a chimeric peptide construct comprises an eIF4E binding domain that is derived from an eIF4E binding protein such as, for example, 4EBP1, 4EBP2, or 4EBP3. A preferred eIF4E binding domain may be selected from the group comprising SEQ ID NO:2, SEQ ID NO:3, SEQ ID NO:4, and SEQ ID NO:5.

Certain embodiments of the present invention provide a chimeric peptide construct comprising a cytoplasmic delivery domain which may improve the retention within the cytoplasm of a cell for a construct in which it is comprised. In these embodiments, a preferred cytoplasmic delivery domain may be an influenza virus hemagglutinin-2 (HA-2) sequence, a photosensitizer, or a melittin-derived peptide.

In some aspects, the present invention is directed to a chimeric peptide construct comprising a cell-penetrating domain which may improve the efficiency of cellular entry by a construct in which it is comprised. An exemplary cell-penetrating domain is a peptide derived from the transactivating (TAT) protein of the human immunodeficiency virus (HIV). Other suitable penetration peptides are known in the art and may be included in a chimeric peptide construct of the instant invention.

Select embodiments of the present invention provide a chimeric peptide construct comprising a cell-targeting domain and an eIF4E binding domain. In some embodiments, a chimeric peptide construct comprises either a cytoplasmic delivery domain or a cell-penetrating domain in addition to a cell-targeting domain and an eIF4E binding domain. In other embodiments of the present invention, a chimeric peptide construct comprises a cytoplasmic delivery domain, a cell-penetrating domain, a cell-targeting domain, and an eIF4E binding domain

In some aspects, the present invention is directed to a chimeric peptide construct comprising a cell-penetrating domain and an eIF4E binding domain. In other aspects, a chimeric peptide construct comprises a cytoplasmic delivery domain in addition to a cell-penetrating domain and an eIF4E binding domain. A cell-penetrating domain may be a peptide derived from HIV-TAT and may be, for example, a peptide comprising SEQ ID NO:8. However, a number of other cell penetrating peptide sequences are known in the art and it is proposed that any such peptide sequence may be employed in the practice of the invention.

Some embodiments of the present invention provide a method of treating a condition characterized by abnormal cell proliferation or abnormal cell survival in a subject, wherein the method comprises administering to a subject in need of such treatment, a therapeutically effective amount of a chimeric peptide construct disclosed herein. In some aspects, the condition characterized by abnormal cell proliferation or abnormal cell survival can be an endocrine cancer or a cancer expressing a GnRH receptor. Endocrine cancers may include, but are not limited to, ovarian cancer, breast cancer, prostate cancer, endometrial cancer, cervical cancer, uterine cancer, and pituitary cancer. Cancers expressing a GnRH receptor can include, by way of non-limiting example, an intracranial tumor, a lymphoma, a melanoma, and a squamous cell carcinoma.

In particular embodiments, the present invention provides a method of treating a condition characterized by abnormal cell proliferation or abnormal cell survival, especially an ovarian cancer, wherein a chimeric peptide construct is administered to a subject in need of such treatment. In preferred embodiments, a chimeric peptide construct disclosed herein is administered to the subject by intraperitoneal injection.

An aspect of the present invention provides a pharmaceutical composition comprising one or more pharmaceutically acceptable excipients and a chimeric peptide construct disclosed herein, in particular, a chimeric peptide construct comprising SEQ ID NO:6 or SEQ ID NO:7. A pharmaceutical composition may be used with a method of treating a proliferative or nonproliferative disease as disclosed herein.

Particular embodiments of the present invention provide a method of treating a condition associated with abnormal protein synthesis in a subject, wherein the method comprises administering to a subject in need of such treatment, a therapeutically effective amount of the chimeric peptide construct disclosed herein. Other embodiments of the invention provide method of inhibiting protein synthesis in a GnRH receptor-bearing cell, wherein the method comprises contacting the cell with the chimeric peptide construct disclosed herein to inhibit protein synthesis in the cell.

In multiple embodiments, the present invention provides kits for use with one or more methods disclosed herein. In particular, a kit for treating a condition associated with abnormal cell proliferation or abnormal cell survival is provided, wherein the kit comprises a container, and a metered quantity of a pharmaceutical composition comprising the chimeric peptide construct disclosed herein. A kit may also comprise an applicator, preferred applicators including, but not limited to, a syringe, an intravenous infusion assembly, an intraperitoneal injection assembly, an applicator for topical administration, and an applicator for subcutaneous administration

As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and/or advantages of the present invention will become apparent from the following detailed description. It should be understood that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1G. 4EBP peptides bind eIF4E and inhibit binding of eIF4E to eIF4G. (FIG. 1A) Sequences of peptides comprising residues 49 to 68 of 4EBP1 and 4EBP2. (FIG. 1B) Biotinylated peptides (3 micromoles per liter) were incubated with 250 micrograms of OVCAR-3 cell lysate. Peptides were pulled-down using streptavidin-agarose. Precipitates (P) and supernatants (S) were analyzed by Western blot using eIF4E antibody. In (FIG. 1C), immunoprecipitation was performed using eIF4E antibody, and Western blot performed using eIF4G antibody. (FIG. 1D) Sequences of wild-type and mutant 4EBP peptides fused to TAT (mutant residues are highlighted). (FIG. 1E) OVCAR-3 cells were treated with biotinylated peptides (3 micromoles per liter, final concentration) for 16 hours. Internalized peptides were detected by staining fixed cells with Texas Red-conjugated streptavidin. Nuclei were visualized by staining with 4′,6-Diamidino-2-Phenylindole (DAPI). (FIGS. 1F and 1G) OVCAR-3 cell lysates were incubated with peptides as described for FIG. 1B. In (FIG. 1F), immunoprecipitation was performed using streptavidin-agarose and Western blot performed using eIF4E antibody. In (FIG. 1G), immunoprecipitation was performed using eIF4E antibody, and Western blot performed using eIF4G antibody.

FIGS. 2A-2F. Inhibition of cap-dependent translation and cell growth by TAT-4EBP fusion peptides. (FIG. 2A) Representation of the pIRES-DualLuc bicistronic construct containing Renilla Luciferase (R-Luc) and firefly luciferase (F-Luc) genes. In (FIG. 2B), R-Luc and F-Luc activities were assayed in ES-2-DualLuc cells following treatment for 24 hours with rapamycin and for 3 hours with cycloheximide with the indicated concentrations of these drugs, and expressed relative to the respective luciferase activities in cells incubated without drug. Cell viability was not affected by rapamycin and cycloheximide under these conditions. In (FIG. 2C), R-Luc and F-Luc activities were assayed in ES-2-DualLuc cells following treatment for 6 hours with the indicated concentrations of peptides, and expressed relative to the respective luciferase activities in cells incubated without peptide. Shown are results of triplicate assays performed independently two times, and the statistical significance of differences in relative R-Luc and F-Luc activities in cells treated with peptide at a concentration of 10 micromoles per liter. (FIG. 2D) Viability of OVCAR-3 cells at 3 days after addition of the indicated concentrations of peptides, expressed relative to viability of cells incubated without peptide. Shown are results of triplicate assays performed in two independent experiments, and statistical significances of differences in viability of cells treated with TAT-4EBP1-WT vs. TAT-4EBP1-MT, and of cells treated with TAT-4EBP2-WT vs. TAT-4EBP2-MT at a concentration of 10 micromoles per liter. (FIG. 2E) OVCAR-3 cells at 3 days after peptide addition (10 micromoles per liter) viewed under phase-contrast microscopy. (FIG. 2F) Flow cytometric analysis of staining of terminal deoxynucleotidyl transferase-mediated dUTP nick end labeling (TUNEL) in OVCAR-3 cells at 3 days after peptide addition (10 micromoles per liter). Cells incubated for 3 days in the absence of peptide in medium with and without serum are included as controls.

FIGS. 3A-3H. Uptake, binding and inhibitory effects of [DLys6]GnRH-4EBP fusion peptides. (FIG. 3A) Sequences of [DLys6]GnRH, and wild-type and mutant 4EBP1 peptides fused to the agonist. (FIG. 3B) Western blot of GnRH-RI levels in OVCAR-3, ES-2, MDA-MB-231, BJ, HUVEC and SKOV3 cells. (FIG. 3C) Detection of biotinylated peptides in OVCAR-3 and SKOV3 by Texas Red-conjugated streptavidin at 16 hours after peptide addition (3 micromoles per liter). In (FIGS. 3D and 3E), biotinylated peptides were incubated with OVCAR-3 cell lysate as described in FIG. 1. In (FIG. 3D), peptide was pulled down using streptavidin-agarose. Precipitates (P) and supernatants (S) were analyzed by Western blot using eIF4E antibody. In (FIG. 3E), immunoprecipitation was performed using eIF4E antibody, and Western blot performed using eIF4G antibody. (FIG. 3F) R-Luc and F-Luc activities were assayed in ES-2-DualLuc cells at 6 hours after addition of the indicated concentrations of peptides, and are expressed relative to the respective luciferase activities in cells incubated without peptide. Shown are results of triplicate assays performed independently two times, and the statistical significance of differences in relative R-Luc and F-Luc activities in cells treated with peptide at a concentration of 10 micromoles per liter (FIG. 3G) Cells of GnRH-RI-positive and -negative lines were treated with the indicated concentrations of peptides. Viability at 3 days after peptide addition is expressed relative to viability of cells incubated with no peptide. Shown are results of triplicate assays performed in two independent experiments, and statistical significances of differences in viability of cells treated with wild-type vs. mutant [DLys6]GnRH-4EBP1 peptides at a concentration of 10 micromoles per liter. (FIG. 3H) ES-2 and parental SKOV3 cells at 3 days after peptide addition (30 micromoles per liter) viewed under phase-contrast microscopy.

FIGS. 4A-4C. Anti-tumor activity of [DLys6]GnRH-4EBP1-WT peptide in a xenograft model of EOC. Female nude mice were inoculated i.p. with one million ES-2-GFP cells. At Day 9 after tumor cell inoculation, one group of mice (n=5) was sacrificed (pre-treatment). At Day 9, other groups of mice were administered by i.p. injection with saline (n=11), [DLys6]GnRH (n=11) or [DLys6]GnRH-4EBP1-WT (n=11) at a dose of 3.0 nanomoles per gram body weight Saline or peptide was administered every 2 days thereafter for 11 days (6 doses in total), and mice were sacrificed on Day 20. (FIG. 4A) Visualization under a fluorescence stereoscope of i.p. tumors in pre-treatment mice (at Day 9) including implants on the omentum (ome), and in mice treated with saline, with agonist alone and with [DLys6]GnRH-4EBP1-WT (at Day 20). (FIG. 4B) Hematoxylin eosin-stained sections of tissues of pre-treatment mice (at Day 9) and of saline- and peptide-treated mice (at Day 20). Tumors attached to the omentum extensively involved the pancreas in saline- and agonist-treated mice. Also shown are tumors attached to the broad ligament and mesentery. Bar, 200 micrometers. (FIG. 4C) Quantification of i.p. tumor burden in pre-treatment and treated mice that completed the regimen, expressed as the average percent of area of fluorescence within the abdominal cavity in captured images, and volume of ascites. Median values of tumor burden and ascites volume for each group and statistical significance of differences between groups are indicated.

FIG. 5. Effect of peptides on reporter RNA levels. ES-2-DualLuc cells were incubated without peptide or treated with the indicated peptides for 6 hours at a final concentration of 10 micromoles per liter. Northern blot analysis of DNase I-treated RNA isolated from these cells was performed using a ³²P-labeled DNA fragment containing the R-Luc and F-Luc genes to detect the bicistronic RNA. Reprobing of the blot with labeled actin cDNA is included as a control.

FIGS. 6A-6B. Stability of [DLys6]GnRH-4EBP1 peptide. Biotinylated peptide was added to McCoys' 5A medium containing 10% fetal bovine serum (FBS), and to human serum. Samples were immediately withdrawn (0 hours). The remaining mixtures were incubated at 37° C., and samples taken thereafter at 1, 3, 6, 12 and 24 hours. In (FIG. 6A), samples were analyzed by Western blot using horseradish peroxidase-conjugated strepatavidin. In (FIG. 6B), samples of peptide incubated in serum for 0, 12 and 24 hours were analyzed by Matrix Assisted Laser Desorption Ionization-Time of Flight (MALDI TOF) mass spectrometry. Peaks corresponding to intact peptide (mass: 3990) are indicated. Peaks of 1256, 1684 and 3384 in mass are likely to represent degradation products of the peptide.

FIGS. 7A-7B. Cellular uptake of [DLys6] GnRH-4EBP1 peptide. Parental ES-2 (FIG. 7A) and OVCAR-3 (FIG. 7B) cells were incubated with carboxyfluorescein (FAM)-conjugated [DLys6]GnRH-4EBP1-WT peptide (shown in green) for 1 hour at 37° C. Cells were fixed and stained with antibodies to early endosomal antigen 1 (EEA1), lysosomal associated membrane protein 2 (LAMP2) and lysosomal associated membrane protein 1 (LAMP1) (shown in red).

FIGS. 8A-8D. Effect of [DLys6] GnRH-4EBP1 peptide in SKOV3 cells stably expressing GnRH-RI. (FIG. 8A) Western blot analysis of GnRH-RI in parental SKOV3 cells and SKOV3 cells transfected with GnRH-RI. In (FIG. 8B), SKOV3+GnRH-RI cells were treated with biotinylated peptides at a final concentration of 10 micromoles per liter or incubated without peptide. At 24 hours after treatment, lysates were prepared from cells. Peptide was pulled-down using streptavidin-agarose. Precipitates and supernatants were analyzed by Western blot using eIF4E antibody. (FIG. 8C) parental SKOV3, SKOV3+GnRH-RI and ES-2 cells were treated with the indicated peptides (10 micromoles per liter) or incubated without peptide. At 24 hours after treatment, lysates were prepared from cells. Immunoprecipitation was performed using eIF4E antibody, and Western blot of precipitates was performed using eIF4G antibody. Western blot of whole cell lysates of treated cells was performed using antibodies to eIF4G, eIF4E and actin. (FIG. 8D) TUNEL-staining of parental SKOV3 and SKOV3+GnRH-RI cells at 3 days following treatment with peptide at a final concentration of 30 micromoles per liter.

FIGS. 9A-9C. Fusion of 4EBP1 peptide to mutant [DLys6]GnRH agonist. (FIG. 9A) Sequences of wild-type 4EBP1 peptides fused to wild-type [DLys6]GnRH and to mutant agonist containing a His to Gln substitution at position 2. (FIG. 9B) OVCAR-3 cells were treated with biotinylated peptides for 16 hours at a final concentration of 3 micromoles per liter. Internalized peptides were detected by staining fixed cells with Texas Red-conjugated streptavidin. Nuclei were visualized by DAPI staining (FIG. 9C) Viability of OVCAR-3 cells following treatment with the indicated concentrations of peptides for 3 days, as measured by crystal violet staining and expressed relative to viability of cells incubated without peptide. Shown are results of triplicate assays performed in two independent experiments, and statistical significance of differences in viability of cells treated with [DLys6]GnRH-4EBP1-WT vs. MT-[DLys6]GnRH-4EBP1-WT at a concentration of 10 micromoles per liter

FIG. 10. Activation of GnRH-RI signal transduction pathway in response to peptides. ES-2 cells were treated with [DLys6]GnRH agonist and with [DLys6]GnRH-4EBP1-WT peptide for 0, 10, 30, 60 and 180 minutes at a final concentration of 10 micromoles per liter. Western blot of whole cell lysates of treated cells was performed using Abs to phosphorylated ERK1/2, p38 and JNK.

FIGS. 11A-11B. TUNEL-staining of tissues of saline- and peptide-treated mice. TUNEL-staining was performed on tissues collected from mice sacrificed at Day 20, following treatment with saline, [DLys6]GnRH and [DLys6]GnRH-4EBP1-WT. Shown are normal tissues of the brain, heart, lung, liver, breast, pancreas and ovary, and tumor implants on the omentum. Bar, 60 micrometers. Pancreatic and ovarian sections show normal pancreatic (P) and ovarian (O) tissues plus adjacent tumor (T). Also shown are normal tissues of the pituitary and bone marrow. Bar, 20 micrometers

FIGS. 12A-12C. Serum luteinizing hormone (LH) levels and serum antibody reactivity. (FIG. 12A) LH levels in serum collected from mice at Day 20 following treatment with saline, [DLys6]GnRH and [DLys6]GnRH-4EBP1-WT. Reactivity of mouse sera to (FIG. 12B) ES-2 cell lysate and to (FIG. 12C) [DLys6]GnRH-4EBP1-WT peptide. The lower level of serum antibody reactivity to tumor cell lysate seen in mice treated with [DLys6]GnRH-4EBP1-WT peptide might be associated with the reduced tumor burden in these mice.

FIG. 13. Cellular uptake of [DLys6] GnRH-4EBP1 peptide without fixation. ES-2 cells were preincubated with FAM-conjugated [DLys6]GnRH-4EBP1-WT peptide for 30 min on ice, washed five times in cold medium to remove unbound peptide and then incubated in peptide-free medium for 1 hour at 37° C. Cells were directly viewed without fixation by fluorescence microscopy (peptide shown in green) and by light microscopy (brightfield).

FIGS. 14A-14B. Specificity of [DLys6]GnRH-4EBP1 fusion peptides. (FIG. 14A): Cells of GnRH-RI-positive (LnCaP, A2780) and -negative (COS, HeLa) lines were treated with the indicated concentrations of peptides. Viability of cells at 3 days after peptide addition was measured by crystal violet staining and is expressed relative to viability of cells incubated with no peptide. Shown are statistical significances of differences in viability of cells treated with wild-type vs. mutant 4EBP1 peptides fused to [DLys6]GnRH at a concentration of 10 micromoles per liter. (FIG. 14B): ES-2 cells were treated for 2 days with wild-type and mutant 4EBP1 peptides fused to [DLys6]GnRH at a final concentration of 10 micromoles per liter. Staining of cells with FITC-Annexin V and PI was analyzed by flow cytometry.

FIG. 15. Response of AKT pathway to [DLys6]GnRH-4EBP1 fusion peptides. ES-2 cells were treated with no peptide or with [DLys6]GnRH, [DLys6]GnRH-4EBP1-WT or [DLys6]GnRH-4EBP1-MT at a final concentration of 10 micromoles per liter for 24 hours. Western blot of whole cell lysates of cells was performed using Ab to phosphorylated AKT. Lysate of ES-2 cells that were stimulated with epidermal growth factor (EGF) (100 nanograms per milliliter) for 30 min was included as a positive control for phosphorylated AKT.

### DESCRIPTION OF ILLUSTRATIVE EMBODIMENTS

The present invention is based, in part, on the development by the inventor of chimeric peptide constructs for targeted inhibition of protein synthesis at the step of translation initiation and methods of use thereof in the treatment of proliferative and non-proliferative diseases. The current invention provides a distinct advantage in cell-specific targeting as this feature avoids the undesirable inhibition of protein synthesis in normal cells observed with other, non-specific inhibitors of protein synthesis. The chimeric peptide constructs disclosed herein and methods of using same to treat proliferative and non-proliferative diseases involving abnormal protein synthesis represent a novel, targeted approach of potential significance for these diseases.

### I. EIF4E and Translation Initiation

Initiation of mRNA translation is an essential step in the process of protein synthesis and is tightly regulated by the eukaryotic translation initiation factor 4E (eIF4E) that binds the 5′ cap structure of mRNA. Interaction of eIF4E with the scaffolding protein eIF4G delivers the mRNA to the 43S pre-initiation complex that scans the 5′-untranslated region (5′-UTR) to reveal the initiation codon and triggers ribosome engagement (Mamane et al., 2004; Proud, 2007). Translation initiation is inhibited when eIF4E is bound to three related proteins called the 4EBPs (i.e. 4EBP1, 4EBP2 and 4EBP3). Phosphorylation of the 4EBPs by mTOR, which is activated by the phosphatidylinositol 3-kinase/AKT signaling pathway, releases the 4EBPs from eIF4E. Once free from the 4EBPs, eIF4E is able to bind eIF4G (Mamane et al., 2004; Proud, 2007). The interactions of eIF4E with the 4EBPs and with eIF4G have been extensively studied (Tomoo et al., 2005; Marcotrigiano et al., 1999; Fletcher et al., 1998).

The rate of translation initiation is primarily governed by the level of free eIF4E in the cell. Levels of free eIF4E are commonly elevated in a wide variety of tumors resulting either from overexpression of eIF4E or from activation of the phosphatidylinositol 3-kinase/AKT signaling pathway (Mamane et al., 2004; De Benedetti and Graff, 2004). Moreover, elevated levels of eIF4E have been demonstrated to promote tumorigenesis (Lazaris-Karatzas et al., 1990; Ruggero et al., 2004). Elevated levels of free eIF4E are thought to promote tumorigenesis by selectively enabling translation of mRNAs that contain long, highly structured 5′-UTRs—a common feature of mRNAs encoding growth and survival factors (Mamane et al., 2004; De Benedetti and Graff, 2004). This preferential enhancement of translation has been demonstrated for mRNAs that encode many growth and survival factors, including vascular endothelial growth factor, ornithine decarboxylase and survivin (Kevil et al., 1996; Rousseau et al., 1996; Mamane et al., 2007). eIF4E also enhances cyclin D1 protein levels by stimulating nuclear export of cyclin D1 mRNA (Rousseau et al., 1996).

Certain embodiments of the present invention provide a chimeric construct comprising an eIF4E binding domain derived from a 4EBP protein. In particular embodiments, an eIF4E binding domain is a 4EBP peptide comprising a sequence which corresponds to, for example, amino acids 49-68 of 4EBP1, 4EBP2, 4EBP3, or a 4EBP consensus sequence. In other embodiments, an eIF4E binding domain is a 4EBP peptide comprising SEQ ID NO:2, SEQ ID NO:3, SEQ ID NO:4, and SEQ ID NO:5. In still other embodiments, an eIF4E binding domain is a mutant, analog, or fragment of a 4EBP peptide which is capable of binding eIF4E, inhibiting the eIF4E-eIF4G interaction, and inhibiting translation initiation in a cell.

### II. GnRH Receptor Targeting

The gonadotropin-releasing hormone, or GnRH, is a hypothalamus-derived decapeptide which stimulates the pituitary gland to produce gonadotropins that control gonadal steroidogenesis (Millar et al., 2004; Harrison et al., 2004). GnRH transmits its signal via two specific G-protein coupled receptors, GnRH-RI and GnRH-RII. In particular, the GnRH-RI is known to signal through the mitogen-activated protein kinase (MAPK) cascades, and is involved in the inhibition of cell proliferation in a variety of cell types. GnRH-RI is overexpressed in 80% of ovarian cancers and is also widely expressed in breast, endometrial and prostate cancers (Harrison et al., 2004; Imai et al., 1994).

GnRH-RI has been the target of drug development efforts, with the use of GnRH agonists to treat endometriosis, uterine fibroids, infertility, precocious puberty, and prostate cancer (Millar et al., 2004; Harrison et al., 2004). While the response rates of some cancer patients to GnRH agonists have been only modest (Adelson and Reece, 1993), the use of GnRH-RI as a cell-surface molecule to which other chemotherapeutics may be targeted has shown promise. GnRH-RI-targeting by GnRH agonists conjugated to doxorubin, camptothecin and cytotoxic peptides has been demonstrated in several preclinical studies (Miyazaki et al., 1997; Dharap et al., 2005; Nechushtan et al., 1997; Chandna et al., 2007).

In certain embodiments, a chimeric construct of the present invention comprises a cell-targeting domain which is a GnRH-RI binding domain. In particular embodiments, a GnRH-RI binding domain is a peptide or an analog of a peptide derived from GnRH wherein one or more amino acids is a D-isomer. A GnRH-RI binding domain may also be any analog, mutant, or fragment of a GnRH peptide which specifically binds a GnRH receptor. In particular embodiments, a GnRH-RI binding domain comprises SEQ ID NO:1.

### III. Cell Penetration and Cytoplasmic Delivery

Cell-penetrating peptides or protein transduction domains (PTDs) are peptides that have the ability to efficiently cross cellular membranes, either alone or in association with molecular cargo. Several naturally occurring PTDs are known, including those from the human immunodeficiency virus transactivation protein (HIV-TAT) and Drosophila antennapedia, as well as synthetic CPPs, such as octa-lysine and nona-arginine. While the precise mechanism(s) of cellular entry for individual CPPs may vary, it is likely that uptake is mediated, at least in part, through endocytosis.

In certain embodiments, the present invention relates to chimeric peptide constructs that feature an eIF4E binding domain and a cell-penetrating domain which improves cell penetration for the chimeric peptide construct in which it is comprised. Cell penetration may, for example, be improved for a chimeric peptide construct disclosed herein by incorporating into the construct a peptide derived from the transactivation protein of the human immunodeficiency virus (HIV-TAT). In some embodiments, a cell-penetrating domain derived from HIV-TAT will comprise at least SEQ ID NO:8. In other embodiments, a cell-penetrating domain is any moiety which enhances penetration of a chimeric peptide construct in which it is comprised.

Without wishing to be limited to a particular theory, it is possible that a chimeric peptide construct disclosed herein can enter a cell by endocytosis. Peptides and other biomolecules that enter cells through this mechanism can become sequestered and subsequently degraded in endosomal compartments, thus failing to access the cell interior and their points of action. A number of cytoplasmic delivery vehicles are known in the art and are suitable for inclusion in a chimeric peptide construct disclosed herein. A cytoplasmic delivery domain may be, for example, a viral or bacterial peptide or analog thereof, a toxin, a photosensitizer, or a synthetic cell-penetrating peptide (CPP). A few endosomolytic domains have been characterized in viral and bacterial proteins, and synthetic CPPs have a similar impact on both endosomal and cell exterior membranes (Michiue et al., 2005). A number of pore-forming or membrane-permeating toxins are known such as, for example, melittin, a naturally occurring, amphipathic CPP derived from the venom of the European honey bee Apis mellifera (Lavignac et al., 2005; Raghuraman et al., 2007). Photochemical internalization is a more recent approach to endosomal release induction in which a photosensitizer, either chemically attached or co-administered, induces photochemical rupture of endosomes resulting in the cytoplasmic delivery of peptides and small molecules (Shiraishi et al., 2006; Berg et al., Caruso et al., 2004; Shiah et al., 2000).

Accordingly, certain embodiments of the present invention provide a chimeric peptide construct which comprises a GnRH-RI binding domain, an eIF4E binding domain, and a cytoplasmic delivery domain. A cytoplasmic delivery domain may be a peptide or small molecule that enhances cytoplasmic delivery of the chimeric peptide construct in which it is comprised. By way of non-limiting example, a chimeric peptide construct, targeted to a GnRH receptor-bearing cell and endocytosed with the receptor following its binding, may accumulate somewhat in endosomal structures, while the same chimeric peptide construct, additionally comprising a cytoplasmic delivery domain, is released more readily into the cell interior.

Any chemical entity known in the art which optimizes the release of peptides or proteins into the cytoplasm of a cell can be included as a cytoplasmic delivery domain of the instant invention. In an aspect, a cytoplasmic delivery domain can be a peptide derived from the Influenza virus hemagglutinin-2 protein (HA-2). By way of nonlimiting example, an HA-2-derived cytoplasmic delivery domain can comprise at least SEQ ID NO:11. In some embodiments, a cytoplasmic delivery domain is a synthetic CPP, such as octalysine or nona-arginine. In other embodiments, the cytoplasmic delivery domain is an analog of melittin or is an analog, mutant, or fragment derived from melittin (SEQ ID NO:12). A melittin-derived cytoplasmic delivery domain can comprise, for example, SEQ ID NO:13. In still other embodiments, the cytoplasmic delivery domain is a photosensitizer, such as aluminum phthalocyanine disulfonate (AlPcS2a), mesochlorin (me6), or N-aspartyl chlorin e6 (NPe6).

### IV. Chimeric Peptide Constructs

In select embodiments, the present invention provides a chimeric peptide construct that comprises a cell-targeting domain and an eIF4E binding domain, and may also comprise one or more of a cell penetrating domain and a cytoplasmic delivery domain. A chimeric peptide construct can comprise, for example, a sequence selected from the group comprising SEQ ID NO:6 and SEQ ID NO:7.

A chimeric peptide construct of the present invention may be comprised of two or more domains disclosed herein in any configuration that allows the required biological functions of each domain to be retained by the construct in which it is comprised. By way of nonlimiting example, a chimeric peptide construct comprising a cell-targeting domain (GnRH), an eIF4E-binding domain (4EBP), and a cytoplasmic delivery domain (CDD) can have an N- to C-terminal configuration selected from the group comprising GnRH-4EBP-CDD, 4EBP-GnRH-CDD, 4EBP-CDD-GnRH, GnRH-CDD-4EBP, CDD-GnRH-4EBP, and CDD-4EBP-GnRH, provided that the selected configuration retains GnRH and eIF4E binding properties and is efficiently delivered to the cytoplasm.

In particular embodiments, the present invention provides a chimeric peptide construct wherein the cell-penetrating domain is N-terminal with respect to the eIF4E binding domain. A chimeric peptide construct can comprise, for example, a sequence selected from the group comprising SEQ ID NO:9 and SEQ ID NO:10.

In an aspect, a cytoplasmic delivery domain may be comprised in the chimeric peptide construct via peptide linkage or by any other appropriate means of chemical conjugation.

In some embodiments, a chimeric peptide construct is a construct comprising an amino acid sequence of SEQ ID NO:6 or having at least 90%, at least 91%, at least 92%, at least 93%, at least 94%, at least 95%, at least 96%, at least 97%, at least 98%, or at least 99% amino acid sequence identity with SEQ ID NO:6. In some embodiments, a chimeric peptide construct is a construct comprising an amino acid sequence of SEQ ID NO:7 or having at least 90%, at least 91%, at least 92%, at least 93%, at least 94%, at least 95%, at least 96%, at least 97%, at least 98%, or at least 99% amino acid sequence identity with SEQ ID NO:7.

Preferred chimeric peptide constructs are those that are able to bind eIF4E specifically, thereby inhibiting the eIF4E-eIF4G interaction and reducing protein synthesis in a cell. Determining whether a particular chimeric peptide construct can bind eIF4E, interfere with its binding of eIF4G and inhibit protein synthesis in a cell can be accomplished using standard assay methods including, but not limited to, in vitro and cell-free translation assays, single ligand and competitive binding assays, reporter assays, and the like. In addition to specific binding of eIF4E, a preferred chimeric peptide construct is capable of selective binding of a GnRH receptor. Any established methods for assessing receptor binding may be suitable for determining selective binding to a GnRH receptor.

In particular embodiments, a chimeric peptide construct may be modified to be more therapeutically effective or suitable. For example, a chimeric peptide construct may be converted into pharmaceutical salts by reaction with inorganic acids including hydrochloric acid, sulphuric acid, hydrobromic acid, phosphoric acid, etc., or organic acids including formic acid, acetic acid, propionic acid, glycolic acid, lactic acid, pyruvic acid, oxalic acid, succinic acid, malic acid, tartaric acid, citric acid, benzoic acid, salicylic acid, benzenesulphonic acid, and tolunesulphonic acids.

In a further embodiment of the instant invention, a chimeric peptide construct may be modified to contain a fluorescent label or other molecular label such as, for example, biotin. The resulting labeled chimeric peptide construct can be used to identify cells expressing GnRH receptors, to quantify GnRH receptor expression on a cell surface, and to identify, by a competitive assay known in the art, other inhibitors of the eIF4E-eIF4G interaction or other useful GnRH receptor agonists and antagonists.

In other embodiments, the present invention contemplates a chemical derivative of a chimeric peptide construct. “Chemical derivative” refers to a protein having one or more residues chemically derivatized by reaction of a functional side group, and retaining biological activity and utility. Such derivatized proteins include, for example, those in which free amino groups have been derivatized to form specific salts or derivatized by alkylation and/or acetylation, p-toluene sulfonyl groups, carbobenzoxy groups, t-butylocycarbonyl groups, chloroacetyl groups, formyl or acetyl groups among others. Free carboxyl groups may be derivatized to form organic or inorganic salts, methyl and ethyl esters or other types of esters or hydrazides and preferably amides (primary or secondary). Chemical derivatives may include those peptides which contain one or more naturally occurring amino acids derivatives of the twenty standard amino acids. For example, 4-hydroxyproline may be substituted for serine; and ornithine may be substituted for lysine. A chemical derivative may also include a chimeric peptide of the present invention which has been chemically modified by one or more of biotinylation, glycosylation, phosphorylation, myristolyation, carboxylation, or amidation.

A chimeric peptide construct of the present invention may also be modified to contain amino acid substitutions, insertions and/or deletions that do not alter the biological properties of the construct. Such a biologically functional equivalent of a chimeric peptide construct could be a molecule having like or otherwise desirable characteristics, i.e. binding a GnRH receptor, penetrating cells efficiently, accessing the cell cytoplasm, binding eIF4E, preventing the eIF4E-eIF4G interaction, and inhibiting protein synthesis. As a nonlimiting example, certain amino acids may be substituted for other amino acids in a chimeric peptide construct without appreciable loss of interactive capacity, as demonstrated by unchanged binding of eIF4E and/or GnRH-RI and unaltered downstream events, such as protein synthesis. It is thus contemplated that a chimeric peptide construct which is modified in sequence and/or structure but which is unchanged in biological utility or activity remains within the scope of the present invention.

It is also well understood by the skilled artisan that, inherent in the definition of a biologically functional equivalent peptide or analog, is the concept that there is a limit to the number of changes that may be made within a defined portion of the molecule and still result in a molecule with an acceptable level of equivalent biological activity. Biologically functional equivalent peptides are thus defined herein as those peptides in which certain, not most or all, of the amino acids may be substituted. Of course, a plurality of distinct chimeric peptide constructs with different substitutions may easily be made and used in accordance with the invention.

The skilled artisan is also aware that where certain residues are shown to be particularly important to the biological or structural properties of a protein or peptide, e.g., residues in active sites, such residues may not generally be exchanged. This is the case in the present invention, where specific changes in a chimeric peptide construct could render it incapable of binding of eIF4E and/or GnRH-RI and inhibiting protein synthesis, and would result in a loss of utility of the resulting construct for the present invention.

Amino acid substitutions, such as those which might be employed in modifying a chimeric peptide construct are generally based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents.

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues. The amino acids described herein are present in the “L” isomeric form unless otherwise designated as a “D” isomeric form by a small capital “D”, as in SEQ ID NO: 1. Any amino acids disclosed herein as being L isomers may be substituted with an amino acid in the D isomeric form, as long as the desired functional properties set forth herein are retained by the construct. In keeping with standard protein nomenclature abbreviations for amino acid residues are known in the art.

Nonstandard amino acids may be incorporated into peptides by chemical modification of existing amino acids or by de novo synthesis of a peptide. A nonstandard amino acid refers to an amino acid that differs in chemical structure from the twenty standard amino acids encoded by the genetic code. Post-translational modification in vivo can also lead to the presence of a nonstandard or amino acid derivative in a protein. The N-terminal and C-terminal groups of a protein can also be modified, for example, by natural or artificial post-translational modification of a protein. Conservative substitutions are least likely to drastically alter the activity of a protein. A “conservative amino acid substitution” refers to replacement of amino acid with a chemically similar amino acid, i.e. replacing nonpolar amino acids with other nonpolar amino acids; substitution of polar amino acids with other polar amino acids, acidic residues with other acidic amino acids, etc.

### V. Methods of Producing a Chimeric Peptide Construct

A chimeric peptide construct may be obtained from known sources, chemically synthesized, or prepared using recombinant techniques known in the art. In select embodiments, a chimeric peptide construct of the invention may be prepared by chemical synthesis using techniques such as solid phase synthesis (Merrifield, 1964) or synthesis in homogenous solution (Houbenweyl, 1987). Solid phase synthesis is a quick and easy approach to synthesizing peptides and small proteins. The C-terminal amino acid is typically attached to a cross-linked polystyrene resin via an acid labile bond with a linker molecule. This resin is insoluble in the solvents used for synthesis, making it relatively simple and fast to wash away excess reagents and by-products.

In select embodiments, a chimeric peptide construct comprises a cytoplasmic delivery domain which is a chemical entity conjugated to a chimeric peptide. Any conjugation chemistry known in the art can be employed to attach a cytoplasmic delivery domain to a chimeric peptide. A structural link between the domains of a chimeric peptide construct may be a peptide bond or it may be some other chemical linkage that can be formed with minimal compromise to the biological function of either domain. One skilled in the art will understand that the choice of linkage and conjugation method will depend on the chemical nature and sensitivities of the cytoplasmic delivery entity. By way of non-limiting example, a chimeric peptide construct may be prepared by carbodiimide condensation to form peptide bonds, by the SPDP (N-succinimidyl 3-(2-pyridyldithio)propionate) method of linking thiol groups and terminal amino groups, by the SMCC method (succinimidyl-4-(N-maleimido methyl)cyclohexane 1-carboxylate) to form peptide bonds, or by N-hydroxisuccinimide (NHS) activation and coupling.

In some embodiments, a chimeric peptide construct can be a chemically modified peptide construct. A peptide construct modification can include phosphorylation (e.g., on a Tyr, Ser or Thr residue), N-terminal acetylation, C-terminal amidation, C-terminal hydrazide, C-terminal methyl ester, fatty acid attachment, sulfonation (tyrosine), N-terminal dansylation, N-terminal succinylation, tripalmitoyl-S-Glyceryl Cysteine (PAMb 3 Cys-OH) as well as famesylation of a Cys residue. Methods of chemically modifying a peptide in these respects are well known in the art.

The term “analogs” as used herein, extends to any functional, chemical or recombinant equivalent of a chimeric peptide construct which demonstrates at least eIF4E binding and GnRH receptor binding. The term “analog” may also be used herein to refer to a peptide having an amino acid sequence which is at least 90%, at least 95%, at least 96%, at least 97%, at least 98%, or at least 99% or more identical to a chimeric peptide construct disclosed herein, such as, for example, a chimeric peptide construct comprising SEQ ID:NO:6 or SEQ ID NO:7

### VI. Cancer and Other Proliferative Diseases

In one aspect, the present invention provides a method of treating a condition characterized by abnormal cell proliferation or abnormal cell survival in a subject, wherein the method may comprise administering to a subject in need of such treatment, a therapeutically effective amount of a chimeric peptide construct disclosed herein. In particular embodiments, the chimeric peptide construct used to treat a condition characterized by abnormal cell proliferation or abnormal cell survival will be comprised in a pharmaceutical composition with one or more pharmaceutically acceptable excipients.

Administration of an “effective amount” of a chimeric peptide construct is defined as an amount effective, at dosages and for periods of time necessary to achieve the desired result. The effective amount of a chimeric peptide construct may vary according to factors such as the disease state, age, sex, and weight of the subject. Using methods well known in the clinical arts, dosage regima may be adjusted to provide the optimum therapeutic response. For example, several divided doses may be administered daily or the dose may be proportionally reduced as indicated by the exigencies of the therapeutic situation.

The terms “subject” and “patient” are used interchangeably and can refer to any mammal, especially a human.

In some embodiments, a condition associated with abnormal cell proliferation or abnormal cell survival is a cancer, a malignancy or tumor. Specific types of cancers include, without limitation, glioma, gliosarcoma, anaplastic astrocytoma, medulloblastoma, lung cancer, small cell lung carcinoma, cervical carcinoma, colon cancer, rectal cancer, chordoma, throat cancer, Kaposi's sarcoma, lymphangiosarcoma, lymphangioendotheliosarcoma, colorectal cancer, endometrium cancer, ovarian cancer, breast cancer, pancreatic cancer, prostate cancer, renal cell carcinoma, hepatic carcinoma, bile duct carcinoma, choriocarcinoma, seminoma, testicular tumor, Wilms' tumor, Ewing's tumor, bladder carcinoma, angiosarcoma, endotheliosarcoma, adenocarcinoma, sweat gland carcinoma, sebaceous gland sarcoma, papillary sarcoma, papillary adenosarcoma, cystadenosarcoma, bronchogenic carcinoma, medullar carcinoma, mastocytoma, mesothelioma, synovioma, melanoma, leiomyosarcoma, rhabdomyosarcoma, neuroblastoma, retinoblastoma, oligodentroglioma, acoustic neuroma, hemangioblastoma, meningioma, pinealoma, ependymoma, craniopharyngioma, epithelial carcinoma, embryonic carcinoma, squamous cell carcinoma, base cell carcinoma, fibrosarcoma, myxoma, myxosarcoma, liposarcoma, chondrosarcoma, osteogenic sarcoma, leukemia, and the metastatic lesions secondary to these primary tumors.

In general, any neoplastic lesion, including granuloma, may be treated according the present invention. In particular embodiments, the present invention may be used to treat an endocrine cancer such as ovarian, endometrial, breast, prostate, cervical, or uterine, and may also be used to treat cancers in which cells are expressing a GnRH receptor including, but not limited to, malignancies of the brain, the skin, and the lymphatic system.

In some embodiments, the chimeric peptide construct administered to a subject for treatment of a condition associated with abnormal cell proliferation or abnormal cell survival is a construct which comprises two or more domains selected from the group comprising a GnRH binding domain, an eIF4E binding domain, a cell-penetrating domain, and a cytoplasmic delivery domain. In other embodiments, the chimeric peptide construct administered to a subject for treatment of a condition associated with abnormal cell proliferation or abnormal cell survival is a construct which comprises an eIF4E binding domain, a cell-penetrating domain, and a cytoplasmic delivery domain. In still other embodiments, the chimeric peptide construct comprises SEQ ID NO:6 or SEQ ID NO:7.

Endocrine cancers commonly overexpress GnRH-RI and are among the most challenging forms of cancer to treat successfully. Most forms of endocrine cancer present with few, if any, easily distinguishing symptoms. Only endometrial cancer is regularly associated with symptoms in early stages of the disease. The 5-year survival rates for prostate, breast, and cervical malignancies have all improved due to widespread application of screening tests and self-examinations. However, ovarian cancer remains difficult to diagnose in its early stages, and is the leading cause of death among gynecological cancers in the U.S., with less than 30% of patients surviving for 5-years post-diagnosis. Treatment for most endocrine cancers, especially ovarian cancer, are usually conventional agents that act nonspecifically on normal and malignant tissues, and generally include taxanes, camptothecins, alkaloids, and platinum compounds. These kinds of agents have a wide range of side-effects which stem from unilateral damage to the normal cells and tissues of the patient.

Accordingly, the present invention provides a method of treating a condition associated with abnormal cell proliferation or abnormal cell survival, wherein the condition is an endocrine cancer and wherein the method comprises administering to a subject in need of such treatment a therapeutically effect amount of a chimeric peptide construct disclosed herein. In some embodiments, the endocrine cancer is a prostate cancer or a pituitary cancer. In other embodiments, the endocrine cancer is an endometrial cancer or a cervical cancer. In yet other embodiments, the endocrine cancer is an ovarian cancer or a breast cancer.

The treatment of ovarian cancer is most often complicated by a late-stage diagnosis. This presents fewer treatment options and a less favorable prognosis. Surgical procedures are almost always the first course of action in treating an ovarian malignancy, and are followed by multiple courses of chemotherapy. Intraperitoneal injection of chemotherapeutic agents is now commonly used in treating some forms of ovarian cancer, particularly epithelial ovarian cancer.

Accordingly, in some embodiments of the present invention, a method is provided for treating a condition associated with abnormal cell proliferation or abnormal cell survival, wherein the condition is ovarian cancer and wherein the method comprises administering to a subject in need of such treatment, a therapeutically effect amount of a chimeric peptide construct disclosed herein. In select embodiments, a chimeric peptide construct may be administered by intraperitoneal injection.

In particular embodiments, the present invention provides a method of treating condition associated with a proliferative disorder, wherein the condition is a cancer or a tumor, the cells of which express a GnRH receptor. This method may comprise administering to a subject in need of such treatment, a chimeric peptide construct disclosed herein. In some aspects, the GnRH receptor may be a GnRH-RI, and the GnRH receptor may be expressed at higher levels in the cells of the tumor than in normal cells of the same tissue. Types of cancer which may express GnRH-RI may include, but are not limited to, an intracranial tumor, a lymphoma, a melanoma, and a squamous cell carcinoma.

In an aspect, the present invention can encompass treatment of a metastasized or secondary tumor originating in an ovary, a breast, a prostate, or from a part of a uterus. The cells of a metastasized tumor may continue to express proteins similar to those expressed in the cells of the primary tumor such as GnRH-RI. Thus, a method is provided for treating a metastasized tumor comprising administering to a subject in need of such treatment, an effective amount of a chimeric peptide disclosed herein. In some embodiments, a metastasized tumor is treated using a chimeric peptide construct comprising a GnRH-RI domain, an eIF4E binding domain, and a cytoplasmic delivery domain. In other embodiments, a metastasized tumor is treated by administering a chimeric peptide construct comprising SEQ ID NO:6 and SEQ ID NO:7.

In some embodiments, a condition associated with abnormal cell proliferation or survival is a squamous cell carcinoma or a melanoma, and a method of treating the condition may comprise topical or subcutaneous administration of a chimeric peptide construct disclosed herein. In other embodiments, a condition associated with abnormal cell proliferation or survival is a pituitary cancer, and a method of treating the condition may comprise intravenous infusion and/or intracranial injection of an effective amount of a chimeric peptide construct disclosed herein. In still other embodiments, a condition associated with abnormal cell proliferation or survival is a lymphoma, and a method of treating the condition may comprise intravenous infusion of an effective amount of a chimeric peptide construct disclosed herein.

Certain embodiments of the present invention pertain to methods of treating a condition associated with abnormal cell proliferation or survival, wherein the condition is a benign tumor, and wherein the method comprises administering to a subject in need of such treatment a therapeutically effect amount of a chimeric peptide construct disclosed herein. By way of non-limiting example, a method of treating a benign pituitary adenoma may include intracranial and/or intravenous administration of a chimeric peptide construct comprising SEQ ID NO:6 or SEQ ID NO:7 to a subject in need of such treatment. In some embodiments, the chimeric peptide construct used to treat a benign tumor is a construct comprising a GnRH binding domain, and an eIF4E binding domain. In other embodiments, the chimeric peptide construct used to treat a benign tumor is a construct comprising two or more domains selected from the group comprising a GnRH binding domain, an eIF4E binding domain, a cell-penetrating domain, and a cytoplasmic delivery domain.

### VII. Non-Proliferative Disorders

In one aspect, the present invention provides a method of treating a condition characterized by abnormal protein synthesis, wherein the condition is a nonproliferative disorder or disease. The term “non-proliferative disorder” is intended to include diseases characterized by a loss of cell and/or organ function due to aberrant protein synthesis. A method of treating a non-proliferative disorder may comprise administering to a subject in need of such treatment, a therapeutically effective amount of a chimeric peptide construct disclosed herein. In select embodiments, the chimeric peptide construct used to treat a non-proliferative disorder will be comprised in a pharmaceutical composition with one or more pharmaceutically acceptable excipients.

In some embodiments, the chimeric peptide construct administered to a subject for treatment of a nonproliferative disorder is a construct which comprises two or more domains selected from the group comprising a GnRH binding domain, an eIF4E binding domain, a cell-penetrating domain, and a cytoplasmic delivery domain. In other embodiments, the chimeric peptide construct administered to a subject for treatment of a nonproliferative disorder is a construct which comprises an eIF4E binding domain, a cell-penetrating domain, and a cytoplasmic delivery domain. In still other embodiments, the chimeric peptide construct comprises SEQ ID NO:6 or SEQ ID NO:7.

In multiple embodiments, a chimeric peptide construct of the present invention may be used to treat non-proliferative diseases and disorders, including but not limited to, Alzheimer's disease, Huntington's disease, liver fibrosis, synucleinopathies, and prion diseases. Other non-proliferative disorders will be recognized by the person of ordinary skill in the art, given the benefit of this disclosure.

A number of neurodegenerative diseases feature aberrant synthesis of one or more proteins, the most well known of these being the dysregulation of tau expression in Alzheimer's disease, at least in part, on the translational level. Accordingly, the present invention contemplates treating a condition characterized by abnormal protein synthesis, wherein the condition is a neurodegenerative disease such as, for example, Alzheimer's disease, synucleinopathy, and prion-mediated disease.

Inappropriate synthesis of at least structural proteins, such as collagen, is implicated in fibrotic diseases of the liver (Gao et al., 2006; Biecker et al., 2005). Accordingly, some aspects of the present invention provide for a method of treating a nonproliferative liver disease, such as cirrhosis or fibrosis, wherein the method comprises administering to a subject in need of such treatment, a chimeric peptide construct of the present invention.

### VIII. Binding Dynamics and Protein Translation Inhibition

The treatment methods of the present invention have stemmed, in part, from the observations by the inventor that a GnRH-RI targeted inhibition of protein synthesis at the level of translation initiation may be useful in abrogating abnormal cell proliferation, abnormal cell survival and aberrant protein synthesis in diseased cells without appreciably affecting normal cells. Therefore, contemplated in select embodiments of the present invention is the use of a chimeric construct disclosed herein to inhibit protein synthesis in a cell, and a method for identifying an inhibitor of the eIF4E-eIF4G interaction.

A. Inhibiting Protein Synthesis in a Cell

Certain embodiments of the present invention pertain to methods of inhibiting protein synthesis in a cell. These methods involve contacting a cell with a chimeric peptide construct disclosed herein, and measuring the protein produced by the cell to determine if the amount of protein synthesis by the cell is decreased. In an aspect, the preferential inhibition of protein synthesis can be determined by performing a measurement of protein synthesis in cells as well as a measurement of the binding of eIFE4 to eIF4G, either in situ or in a cell-free experiment.

In select embodiments, a method of inhibiting protein synthesis in a cell is performed using a GnRH receptor-bearing cell, that is a cell which is known or has been demonstrated to express the GnRH receptor on its surface. In these embodiments, cells expressing a GnRH receptor of type I may be contacted with a chimeric peptide construct comprising a GnRH receptor binding domain such as, for example, SEQ ID NO:6 or SEQ ID NO:7.

For the purposes of inhibiting protein synthesis in a cell, a suitable carrier in which to provide a chimeric peptide construct disclosed herein to the cell can be any suitable carrier used for cellular treatment procedures in the art. The carrier can be, for example, a sterile aqueous-based solution, and can comprise a chimeric peptide construct as well as one or more agents selected from agents regularly used in the art which are compatible with cell culture procedures.

The eIF4E binding dynamics, translation initiation and protein synthesis may be assessed using any appropriate methods known in the art. The dynamics of eIF4E binding can be assessed using an appropriate binding assay and/or competitive binding assays and may be performed using a labeled chimeric peptide construct. By way of non-limiting example, the amount of protein produced by an ovarian cancer cell following exposure to a chimeric peptide construct disclosed herein can be assessed using conventional Bradford, Lowry, or fluorescence methods and the amount of a specific protein produced by the cell can be examined using a conventional gene reporter assay, an ELISA assay or other assay using antibody-mediated detection

B. Identifying an Inhibitor of the eIF4E-eIF4G Interaction

A method of identifying an inhibitor of the eIF4E-eIF4G interaction may also be used to assess the strength or dynamics of a peptide construct to eIF4E. The method of performing such an assessment would employ a particular assay system developed and utilized in the art known as a binding assay or a competitive binding assay. In a binding assay, either the candidate inhibitor to be assayed is labeled or the known binding agent is labeled and then a biomolecule of interest, in this case, eIF4E, is contacted with one or more of a labeled candidate inhibitor, a known inhibitor, and a labeled known binding agent. Upon contacting the biomolecule with known and candidate ligands, an assessment is made of properties or activities which are altered by the presence of the candidate inhibitor. By way of non-limiting example, the detected signal from the binding of a labeled chimeric peptide construct to eIF4E may be observed to alter in the presence of eIF4G, indicating that the construct and the eIF4G are competitively binding eIF4E. The same procedure performed with labeled eIF4G and unlabeled construct, wherein the signal from the eIF4E-eIF4G complex is altered, may identify the construct as an inhibitor of the eIF4E-eIF4G interaction. This method may also be useful in comparisons of binding affinity among eIF4E binding partners and candidate inhibitors.

The labels envisioned for use with methods disclosed herein can include, without limitation, radioactive elements, enzymes, chemicals that fluoresce when exposed to ultraviolet light, biotin, and others. A number of fluorescent materials are known and can be utilized as labels. These include, for example, fluorescein, rhodamine, auramine, Texas Red, AMCA blue and Lucifer Yellow.

### IX. Pharmaceutical Compositions

The present invention provides pharmaceutical compositions comprising one or more pharmaceutically acceptable excipients and a chimeric peptide construct disclosed herein for use in treating a condition associated with abnormal cell proliferation and/or abnormal cell survival and/or abnormal protein synthesis and for inhibiting protein synthesis in a cell. The phrase “pharmaceutically acceptable” refers to molecular entities and compositions that do not produce an adverse, allergic or other untoward reaction when administered to a subject, such as, for example, a human, as appropriate.

In an aspect, a pharmaceutical composition of the present invention can comprise a chimeric peptide construct that is a construct disclosed herein or a construct which retains two or more biological properties selected from the group comprising eIF4E binding GnRH receptor binding, effective cell penetration, and effective cytoplasmic localization. A chimeric peptide construct may, for example, comprise SEQ ID NO:6 or SEQ ID NO:7.

A chimeric peptide construct, or its functional analog, modification or derivative, can be administered as the entity as such or as a pharmaceutically acceptable acid- or base-addition salt, formed by reaction with an inorganic acid (such as hydrochloric acid, hydrobromic acid, perchloric acid, nitric acid, thiocyanic acid, sulfuric acid, and phosphoric acid); or with an organic acid (such as formic acid, acetic acid, propionic acid, glycolic acid, lactic acid, pyruvic acid, oxalic acid, malonic acid, succinic acid, maleic acid, and fumaric acid); or by reaction with an inorganic base (such as sodium hydroxide, ammonium hydroxide, potassium hydroxide); or with an organic base (such as mono-, di-, trialkyl and aryl amines and substituted ethanolamines). A selected peptide and any of the derived entities may also be conjugated to sugars, lipids, other polypeptides, nucleic acids and PNA; and function in-situ as a conjugate or be released locally after reaching a targeted tissue or organ.

In some embodiments, a pharmaceutical composition can include, albeit not exclusively, a chimeric peptide construct in association with one or more pharmaceutically acceptable vehicles or diluents, and contained in buffered solutions with a suitable pH and iso-osmotic with the physiological fluids. The pharmaceutical compositions may additionally contain other agents such as immunosuppressive drugs or antibodies to enhance immune tolerance. The term “active ingredient” is used herein to denote a chimeric peptide construct disclosed herein.

A pharmaceutical composition used in accordance with a method of the invention can be an aerosolized powder or liquid, a liquid, a solid or a semisolid and can be formulated in, for example, pills, tablets, creams, ointments, inhalants, gelatin capsules, capsules, suppositories, soft gelatin capsules, gels, membranes, tubelets, solutions or suspensions.

A pharmaceutical composition of the present invention may comprise different types of carriers depending on whether it is to be administered in solid, liquid or aerosol form, and whether it need to be sterile for such routes of administration as injection. A pharmaceutical composition of the invention can be intended for administration to humans or other animals. Dosages to be administered depend on individual needs, on the desired effect and on the chosen route of administration.

In accordance with the present invention, a pharmaceutical composition containing a chimeric peptide construct can be administered intravenously, intradermally, intraarterially, intraperitoneally, intralesionally, intracranially, intraarticularly, intraprostaticaly, intrapleurally, intrasynovially, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularly, orally, topically, by inhalation, infusion, continuous infusion, localized perfusion, via a catheter, via a lavage, in lipid compositions (e.g., liposomes), or by other method or any combination of the forgoing as would be known to one of ordinary skill in the art.

A pharmaceutical composition can be prepared by known methods for the preparation of pharmaceutically acceptable compositions which can be administered to patients, and such that an effective quantity of the active substance is combined in a mixture with a pharmaceutically acceptable vehicle. Suitable vehicles are described, for example, in Remington's Pharmaceutical Sciences (2005), incorporated herein by reference.

In an aspect, a pharmaceutical composition of the present invention can comprise a therapeutically effective amount of a chimeric peptide construct described herein. The phrase “therapeutically effective amount” refers to an amount of a composition required to achieve a desired medical result, in particular, to achieve the treatment of an autoimmune disease. The preparation of therapeutically effective compositions will be known to those of skill in the art in light of the present disclosure, as exemplified by Remington's Pharmaceutical Sciences. Moreover, for animal, and particularly, human administration, it will be understood that preparations should meet sterility, pyrogenicity, general safety and purity standards as required by FDA Office of Biological Standards.

As used herein, “a composition comprising a therapeutically effective amount” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art. Except insofar as any conventional carrier is incompatible with a chimeric peptide construct, its use in the present compositions is contemplated.

The actual required amount of a composition of the present invention administered to a patient can be determined by physical and physiological factors such as body weight, severity of condition, the type of disease being treated, previous or concurrent therapeutic interventions, idiopathy of the patient and on the route of administration. The practitioner of ordinary skill will rely on methods well established in the art to determine the concentration of active ingredient(s) in a composition and appropriate dose(s) for the individual subject.

In certain embodiments, a chimeric peptide construct may comprise at least about 0.01% of a pharmaceutical composition. In other embodiments, a chimeric peptide construct may comprise between about 2% to about 75% of a pharmaceutical composition, or between about 25% to about 60%, for example, and any range derivable therein. In other non-limiting examples, a dose may comprise from about 0.1 mg/kg/body weight to about 1000 mg/kg/body weight or any amount within this range, or any amount greater than 1000 mg/kg/body weight per administration.

In any case, the composition may comprise various antioxidants to retard oxidation of one or more component. Additionally, the prevention of the action of microorganisms can be brought about by preservatives such as various antibacterial and antifungal agents, including, but not limited to parabens (e.g., methylparabens, propylparabens), chlorobutanol, phenol, sorbic acid, thimerosal or combinations thereof.

In embodiments where the composition is in a liquid form, a carrier can be a solvent or dispersion medium comprising, but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, carbohydrates, sodium chloride or combinations thereof.

Sterile injectable solutions are prepared by incorporating a chimeric peptide construct in the required amount of the appropriate solvent with various amounts of the other ingredients enumerated above, as required, followed by filtered sterilization. Generally, dispersions are prepared by incorporating the various sterilized active ingredients into a sterile vehicle which contains the basic dispersion medium and/or the other ingredients.

In the case of sterile powders for the preparation of sterile injectable solutions, suspensions or emulsion, the preferred methods of preparation are vacuum-drying or freeze-drying or lyophilization techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

In some embodiments, a chimeric peptide construct disclosed herein may be administered to the airways of a subject by any suitable means. In particular, a chimeric peptide construct can be administered by generating an aerosol comprised of respirable particles, the respirable particles comprising at least a chimeric peptide construct, which particles the subject inhales. The respirable particles may be liquid or solid. The particles may optionally contain other therapeutic ingredients.

In an aspect, the present invention encompasses combination therapy for condition associated with abnormal cell proliferation or abnormal cell survival wherein a pharmaceutical composition comprises a chimeric peptide construct, one or more pharmaceutically acceptable excipients and one or more other therapeutic agents. In some embodiments, a pharmaceutical composition comprising a chimeric peptide construct and one or more other therapeutic agents can be used in accordance with a method of treating an condition associated with abnormal cell proliferation or abnormal cell survival disclosed herein.

In preferred embodiments, a chimeric peptide construct can be comprised in a pharmaceutical composition with one or more other therapeutic agents including, but not limited to, a lipid, an endosomolytic agent, an antitumor agent, an anti-emetic, an anesthetic, an analgesic, an angiogenesis inhibitor, and an anti-inflammatory agent. In an aspect, a pharmaceutical composition can comprise a chimeric peptide construct disclosed herein, an antitumor agent, an anti-emetic, and an endosomolytic agent. In another aspect, a pharmaceutical composition can comprise an antitumor agent, an anti-emetic, an endosomolytic agent, and a chimeric peptide construct comprising SEQ ID NO:6 or SEQ ID NO:7.

In particular embodiments, prolonged absorption of an injectable composition can be brought about by the use in the compositions of agents delaying absorption, such as, for example, aluminum monostearate, gelatin or combinations thereof.

A pharmaceutical composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

### X. Kits

Certain embodiments of the present invention are generally concerned with kits for treating proliferative diseases and nonproliferative degenerative diseases, or for investigations of binding and signaling behaviors involving GnRH, GnRH receptors and eIF4E.

In select embodiments, the present invention provides a kit for treating a condition associated with abnormal proliferation or abnormal cell survival comprising a container, and a metered quantity of a pharmaceutical composition comprising a chimeric peptide construct and one or more pharmaceutically acceptable excipients disposed therein. A kit for treating a condition associated with abnormal proliferation or abnormal survival may additionally comprise an applicator selected according to the mode of administration of the construct which is appropriate for the condition to be treated. Applicators which may be used in accordance with the present invention can include, but are not limited to, an injection syringe, an intravenous infusion assembly, an intraperitoneal injection assembly, an applicator for topical administration, an applicator for subcutaneous administration, or an applicator for administration by inhalation.

A kit of the present invention may also contain conventional pharmaceutical adjunct materials such as, for example, pharmaceutically acceptable salts to adjust the osmotic pressure, buffers, preservatives, antioxidants, and the like. A kit may provide patient information and/or a dosing instruction insert as appropriate.

In particular embodiments, a kit is provided for treating a condition associated with abnormal proliferation or survival, wherein the condition is ovarian cancer, and wherein the kit comprises a container, a metered quantity of a pharmaceutical composition comprising a chimeric peptide construct disposed therein, and an intraperitoneal injection assembly.

Certain embodiments of the present invention provide a kit for treating a condition associated with abnormal proliferation or survival, wherein the condition is a prostate cancer or a uterine cancer, and wherein the kit comprises a container, a metered quantity of a pharmaceutical composition comprising a chimeric peptide construct disposed therein, and an intravenous infusion assembly or an intraperitoneal injection assembly.

In other embodiments, the present invention provides a kit for treating a condition associated with abnormal proliferation or survival wherein the condition is a squamous cell carcinoma or a melanoma, and wherein the kit comprises a container, a metered quantity of a pharmaceutical composition comprising a chimeric peptide construct disposed therein, and a an applicator for subcutaneous administration. In particular embodiments, a kit for treating a squamous cell carcinoma or a melanoma comprises a container, a metered quantity of a pharmaceutical composition comprising a chimeric peptide construct disposed therein, and an applicator for topical administration.

When reagents and/or components comprising a kit are provided in a lyophilized form (lyophilisate) or as a dry powder, the lyophilisate or powder can be reconstituted by the addition of a suitable solvent. In particular embodiments, the solvent may be a sterile, pharmaceutically acceptable buffer and/or other diluent. It is envisioned that such a solvent may also be provided as part of a kit.

When the components of a kit are provided in one and/or more liquid solutions, the liquid solution may be, by way of non-limiting example, a sterile, aqueous solution. The compositions may also be formulated into an administrative composition. In this case, the container means may itself be a syringe, pipette, topical applicator or the like, from which the formulation may be applied to an affected area of the body, injected into a subject, and/or applied to or mixed with the other components of the kit.

In select embodiments, kits are contemplated which comprise one or more chimeric peptide constructs, one or more containers, one or more reagents for modification of a chimeric peptide construct, and/or one or more labeling or detection reagents. These kits may be used generally for investigating 4EBP dynamics, eIF4E binding properties, inhibition of translation initiation and protein synthesis in select cell populations, GnRH receptor binding and signaling, and the like. In particular embodiments, the labeling or detection reagent can be any reagent used commonly in the art. Exemplary detection reagents may include, but are not limited to, radioactive elements, enzymes, biotin-containing molecules, molecules which absorb light in the UV range, and fluorophores such as fluorescein, rhodamine, auramine, Texas Red, AMCA blue and Lucifer Yellow. In other embodiments, a kit is provided comprising one or more container means and a chimeric peptide construct already labeled with a detection reagent selected from a group comprising a radioactive element, an enzyme, a molecule which absorbs light in the UV range, and a fluorophore.

### EXAMPLES

The following examples are included to demonstrate preferred embodiments of the invention. It should be appreciated by those of skill in the art that the techniques disclosed in the following example represent techniques identified by the applicant to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Tumor Targeting and Inhibitory Activity of Chimeric Peptide Constructs

### Summary

A critical step of protein synthesis involves the liberation of the mRNA cap-binding eukaryotic translation initiation factor 4E (eIF4E) from the 4EBP inhibitory binding proteins, and its engagement to the scaffolding protein eIF4G. eIF4E is a candidate target for cancer therapy because it is overexpressed or activated in many types of tumors and has tumorigenic properties. In this example, 4EBP-based peptides were generated that bind eIF4E, prevent eIF4E from binding eIF4G, and inhibit cap-dependent translation. A cell-specific delivery system was incorporated by fusing 4EBP peptide to an analog of gonadotropin-releasing hormone (GnRH) to target its receptor that is widely overexpressed in ovarian cancer. GnRH agonist-4EBP chimeric peptide was taken up by, and inhibited cap-dependent translation in, GnRH receptor-expressing tumor cells. GnRH-4EBP chimeric peptide inhibited growth of cultures of GnRH receptor-expressing tumor cells, but not of cells that lack the receptor. This chimeric peptide also substantially inhibited growth of i.p. ovarian tumors in mice without significant cytotoxic effects to host tissues. Because ovarian cancer is rarely cured by conventional therapies, GnRH agonist-4EBP chimeric peptide represents a novel targeted agent of potential significance for treating this disease. This chimeric peptide also has application to treatment of other cancers that overexpress GnRH receptor and have elevated levels of eIF4E.

### Materials and Methods

Peptides and other reagents. Peptides were synthesized to >98% purity according to the design described herein by AnaSpec Inc (San Jose, Calif.), and dissolved in water. The following antibodies (Ab) were used: unconjugated eIF4E Ab (BD Biosciences, San Jose Calif.); agarose-conjugated eIF4E Ab (Santa Cruz Biotechnology, Santa Cruz Calif.); eIF4G Ab (Cell Signaling Technology, Danvers Mass.); GnRH-RI Ab (Exalpha Biologicals, Maynard Mass.); actin Ab (Sigma, St. Louis Mo.); secondary Abs (BioRad, Hercules Calif.). Sources of other reagents were as follows: streptavidin-Texas Red (Invitrogen, Carlsbad Calif.); streptavidin-agarose (Sigma); rapamycin (Calbiochem, San Diego Calif.); cycloheximide (Sigma).

Plasmids. The bicistronic construct containing Renilla luciferase (R-Luc) and firefly luciferase (F-Luc) genes separated by the hepatitis C virus (HCV) type 2b internal ribosomal entry site (IRES) (Collier et al., 1998) was provided by Richard Elliott (University of St. Andrews, U.K.). The biscistronic cassette was subcloned into the pCMVScript vector (Stratagene, La Jolla Calif.) to generate the pIRES-DualLuc construct.

Cell lines. Sources of cell lines were as follows: OVCAR-3 and SKOV3 (American Type Culture Collection, Rockville Md.), ES-2 (Patrice Morin, National Institute on Aging, Baltimore Md.), MDA-MB-231, BJ and HUVEC (Gordon Mills, Shiaw-Yih Lin and Lee Ellis, M.D. Anderson Cancer Center, Houston Tex.). The ES-2-DualLuc line was generated by transfecting ES-2 cells with pIRES-DualLuc plasmid using FuGENE6 reagent (Roche, Indianapolis, Ind.), and selecting stably transfected clones using G418 (400 micrograms per milliliter) (Invitrogen).

Immunoprecipitation (IP) and Western blotting. Biotinylated peptides were incubated with 250 micrograms of cell lysate at a final concentration of 3 micromoles per liter for 6 hours at 4° C. To determine binding of peptides to eIF4E, peptides were pulled down using streptavidin-agarose, and Western blot performed using eIF4E Ab. To determine the ability of peptides to block eIF4E from binding eIF4G, immunoprecipitation was performed using agarose-conjugated eIF4E Ab, and Western blot performed using eIF4G Ab. Immunoprecipitation was likewise performed using lysates of cells that had been cultured with addition of peptides at a final concentration of 10 micromoles per liter for 24 h.

Immunofluorescence staining. For detecting peptide uptake, cells were plated in 2-well chamber slides and incubated with biotinylated peptides at a final concentration of 3 micromoles per liter. Cells were permeabilized in methanol for 20 minutes on ice, and washed with phosphate-buffered saline (PBS) containing 1% bovine serum albumin (BSA). Cells were incubated with Texas Red-conjugated streptavidin (1:500) for 1 hour at 4° C., washed with PBS/BSA, stained with DAPI and viewed under a Nikon 80i fluorescence microscope.

Cell-based translation assays. Five thousand ES-2-DualLuc cells were plated per well in 96-well plates in McCoy's 5A medium (Invitrogen) containing 10% FBS. Following attachment, cells were cultured in FBS-free medium overnight, then cultured in medium containing 10% FBS for 6 hours with addition of peptide at final concentrations of 0, 0.3, 1.0, 3.0, 10.0 and 30.0 micromoles per liter. Cells were also incubated with rapamycin for 24 hours and with cycloheximide for 3 hours at concentrations indicated in the text. F-Luc and R-Luc activities in cells were assayed using the Dual-reporter assay kit (Promega, Madison Wis.). Triplicate wells were set up for each assay, and assays were independently performed two times.

Cell viability and TUNEL assays. For cell viability assays, two thousand cells were plated per well in 96-well plates in medium containing 10% FBS. Following attachment, cells were cultured in FBS-free medium overnight, and thereafter cultured in medium containing 10% FBS for 3 days with addition of peptide at final concentrations of 0, 0.3, 1.0, 3.0, 10.0 and 30.0 micromoles per liter. Cells were stained with 0.5% crystal violet solution, and absorbance measured at 570 nm. For TUNEL assays, 2×10⁵ cells were plated in 60 mm dishes, and incubated with or without peptide for 3 days as described above. Cells were fixed in 70% ethanol and the extent of apoptosis was determined by flow cytometric analysis of TUNEL-staining using the APO-BRDU kit (Phoenix Flow Systems, San Diego Calif.). Triplicate wells were set up for each assay, and assays were independently performed two times.

Animal Studies. Animal studies were approved by the Institutional Animal Care and Use Committee. The ES-2 cell line stably expressing enhanced green fluorescent protein (ES-2-GFP) has been described in previous work (Hara et al., 2007). Four-week-old female nude mice were inoculated i.p. at one site in the lower abdomen with 10⁶ ES-2-GFP cells. At Day 9 after tumor cell inoculation, one group of mice (n=5) were killed by CO2 asphyxiation. At Day 9, other groups of mice were administered by i.p. injection with 200 microliters of phosphate-buffered saline (PBS) (n=11), [DLys6]GnRH (n=11) or [DLys6]GnRH-4EBP1-WT peptide (n=11) at a dose of 3.0 nanomoles per gram body weight. Peptides were suspended in PBS and administered at a volume of 200 microliters. PBS or peptide was administered every 2 days thereafter for 11 days (6 doses in total). Mice were sacrificed on Day 20, and ascites harvested. Tumors were visualized under a Leica MZML III fluorescence stereomicroscope equipped with mercury lamp power supply and green fluorescent protein (GFP) filter set. Tumor burden was quantified by measuring areas of fluorescence signals within the abdominal cavity in captured images by using Image Pro Plus 5.0 software (Media Cybernetics, Bethesda, Md.). Slides of paraffin-embedded tissues collected from mice were stained with hematoxylin-eosin. TUNEL-staining of tissue sections was performed using the in situ cell death detection kit (Roche).

Antibodies. The following antibodies were used: EEA1, LAMP 1, LAMP2 (BD Biosciences); phospho-ERK1/2, phospho-p38, phospho-JNK (Cell Signaling Technology); Alexa Fluor 594 anti-mouse IgG (Invitrogen).

RNA analysis. Northern blot analysis was performed using 10 micrograms of total RNA that was isolated from cells using Trizol (Invitrogen) and treated with DNase I. RNA was transferred to Hybond N+ membrane (GE Healthcare), and hybridized to ³²P-labeled DNA probes using ExpressHyb solution (Clontech).

Peptide stability studies. Biotinylated peptide (7 micrograms) was mixed with McCoys' 5A medium containing 10% FBS in a final volume of 140 microliters. A sample of 20 microliters was immediately removed and frozen. The remaining mixture was incubated at 37° C., and 20 microliters samples taken thereafter at 1, 3, 6, 12 and 24 hours. Samples were likewise taken of biotinylated peptide incubated at 37° C. in undiluted serum of an ovarian cancer patient (obtained from the Cooperative Human Tissue Network). Samples were analyzed by Western blot using horseradish peroxidase-conjugated strepatavidin, and by MALDI TOF mass spectrometry using an Applied Biosystems 4700 Proteomic Analyzer.

Plasmids and cell transfection. The human GnRH-RI cDNA clone was purchased from OriGene Technology (Rockville Md.) and subcloned into the pcDNA3.1+ vector (Invitrogen). The resulting construct was used to stably transfect SKOV3 cells to generate the SKOV3+GnRH-RI cell line.

Immunofluorescence staining. ES-2 and OVCAR-3 cells were plated in 2-well chamber slides, and incubated for 30 min on ice in pre-cooled medium with the addition of FAM-conjugated [DLys6]GnRH-4EBP1-WT peptide (synthesized by Anaspec, Inc) at a final concentration of 3 micromoles per liter. Cells were washed in cold medium to remove unbound peptide and then incubated in pre-warmed medium for 1 hour at 37° C. Cells were fixed in 4% paraformaldehyde at 20 min at 4° C., permeabilized using 0.1% Triton X-100 for 15 min, and then blocked in 1% goat serum for 30 min. Cells were stained with Abs to EEA1, LAMP1 and LAMP2 at 1:200 dilution for 1 hour at 4° C., followed by staining using Alexa Fluor 594 anti-mouse IgG (1:1000 dilution) for 30 min. To eliminate the possibility of artifactual intracellular localization caused by fixation, cells were incubated with FAM-conjugated peptide as described above, washed five times with medium, and directly viewed by fluorescence microscopy without fixation. Cells were also incubated with non-fluorescent peptide, stained with FITC-Annexin V (BD Biosciences) and propidium iodide (PI), and analyzed by flow cytometric analysis.

Assay of serum LH levels. Serum was collected from mice at Day 20 following treatment with saline, [DLys6]GnRH and [DLys6]GnRH-4EBP1-WT and assayed for LH levels by radioimmunoassay (Andrew Parlow, National Hormone & Peptide Program, Harbor-UCLA, Torrance Calif.).

Mouse serum antibody assays. Wells in 96-well plates were coated with 50 nanograms of [DLys6]GnRH-4EBP1-WT peptide or with 100 nanograms of ES-2 cell lysate. Serum of saline- and peptide-treated mice (1:100 dilution) was incubated in wells for 2 hours at room temperature. Bound IgG was detected using horseradish peroxidase-conjugated goat anti-mouse IgG, and measured at an optimal density of 450 nm.

Statistical analysis. Data was analyzed using STATISTICA6 software (StatSoft Inc., Tulsa, Okla.). Analysis of data from animal studies was performed by using the nonparametric Mann-Whitney U-test. Statistical significance of differences in cell viability and luciferase activity was calculated by the Student t-test. P values less than 0.05 were considered to be statistically significant.

### Results

Design of 4EBP Peptides that Bind eIF4E and Inhibit Binding of eIF4E to eIF4G

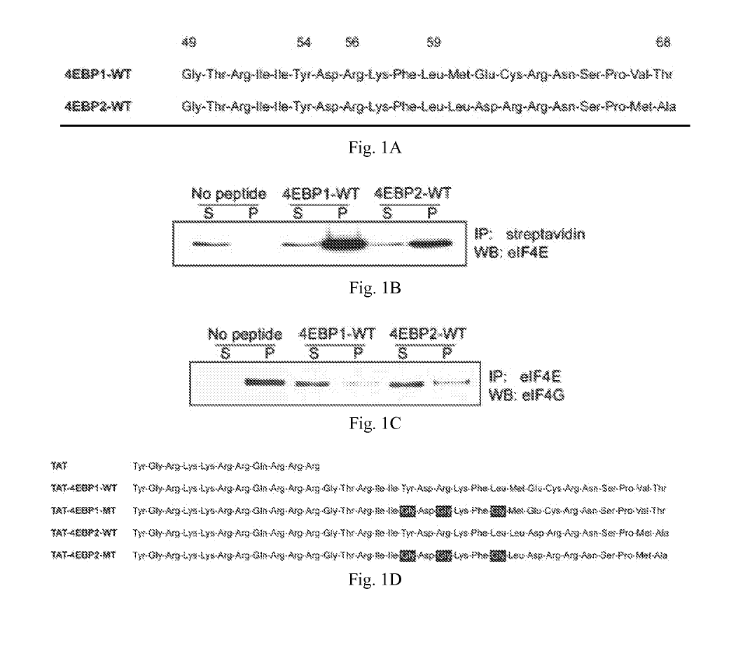

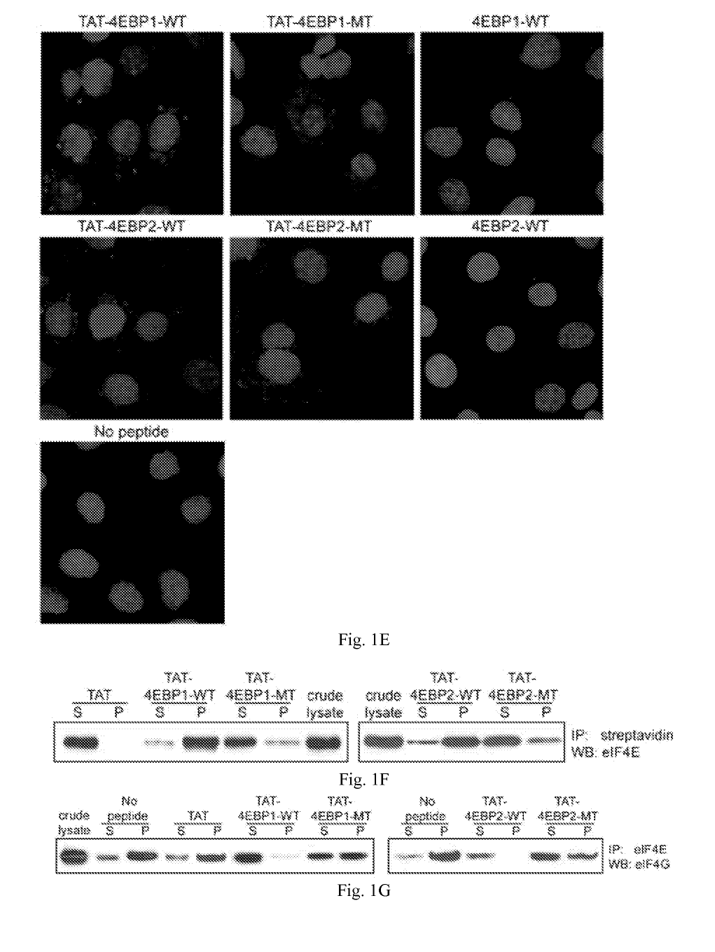

Of the highly related 4EBPs, 4EBP1 is the most characterized. Structural studies have identified the region spanning residues 49 to 68 of 4EBP1 to bind eIF4E (Tomoo et al., 2005; Marcotrigiano et al., 1999; Fletcher et al., 1998). It has also been reported that 4EBPs can bind eIF4E without folded structure (Fletcher et al., 1998). One peptide was synthesized comprising residues 49-68 of 4EBP1 (4EBP1-WT). A second peptide was synthesized containing the corresponding region of 4EBP2 (4EBP2-WT) (FIG. 1A). Both peptides bound to eIF4E in cell extracts of the ovarian cancer cell line OVCAR-3, with 4EBP1-WT demonstrating stronger binding (FIG. 1B). These peptides inhibited eIF4E from binding eIF4G, with 4EBP1-WT demonstrating stronger inhibitory activity (FIG. 1C).

Fusion of TAT to 4EBP Peptides Enables Cellular Uptake and does not Interfere with eIF4E-Binding

To render 4EBP peptides cell-permeable, 4EBP residues were fused to cell-penetrating residues of HIV TAT protein (FIG. 1D). These fusion peptides were water-soluble. TAT-4EBP fusion peptides were taken up by OVCAR-3 cells, whereas 4EBP peptides that lacked TAT were not internalized (FIG. 1E). Fusion of TAT to 4EBP peptides did not interfere with their ability to bind eIF4E (FIG. 1F), and to inhibit eIF4E from binding eIF4G (FIG. 1G). The conserved residues Tyr-54, Arg-56 and Leu-59 of 4EBP proteins are important for binding to eIF4E through hydrophobic and electrostatic interactions, and hydrogen bonds (Tomoo et al., 2005; Marcotrigiano et al., 1999). Mutant TAT-4EBP1 and mutant TAT-4EBP2 fusion peptides were generated in which these three residues were substituted by Gly (FIG. 1D). These mutations substantially impaired the ability of TAT-4EBP peptides to bind eIF4E and to inhibit eIF4E from binding eIF4G (FIGS. 1F,G).

### Inhibition of Cap-Dependent Translation and Cell Growth by TAT-4EBP Fusion Peptides

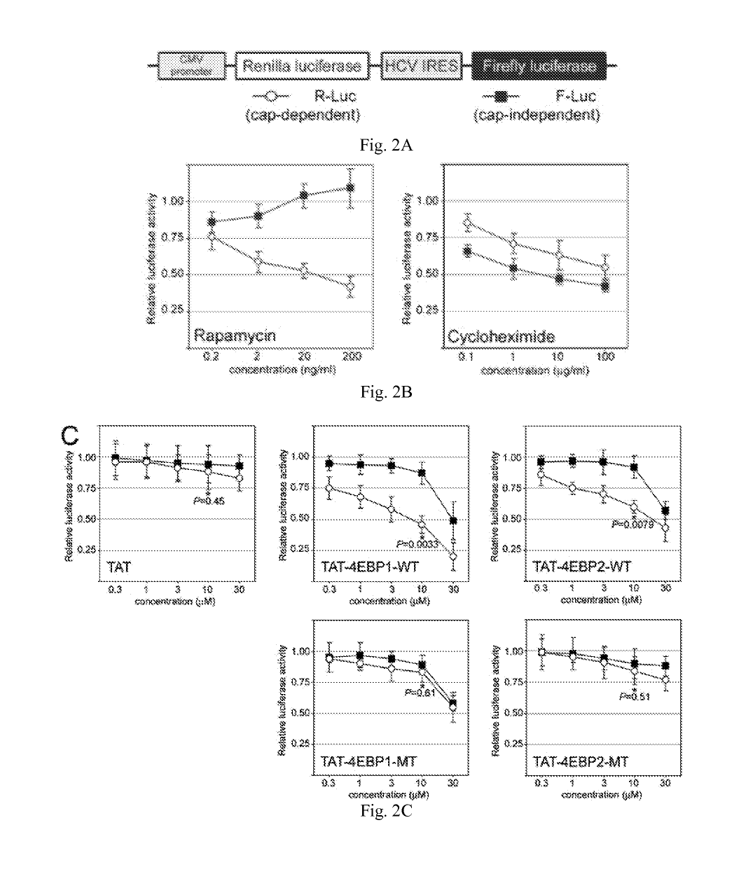

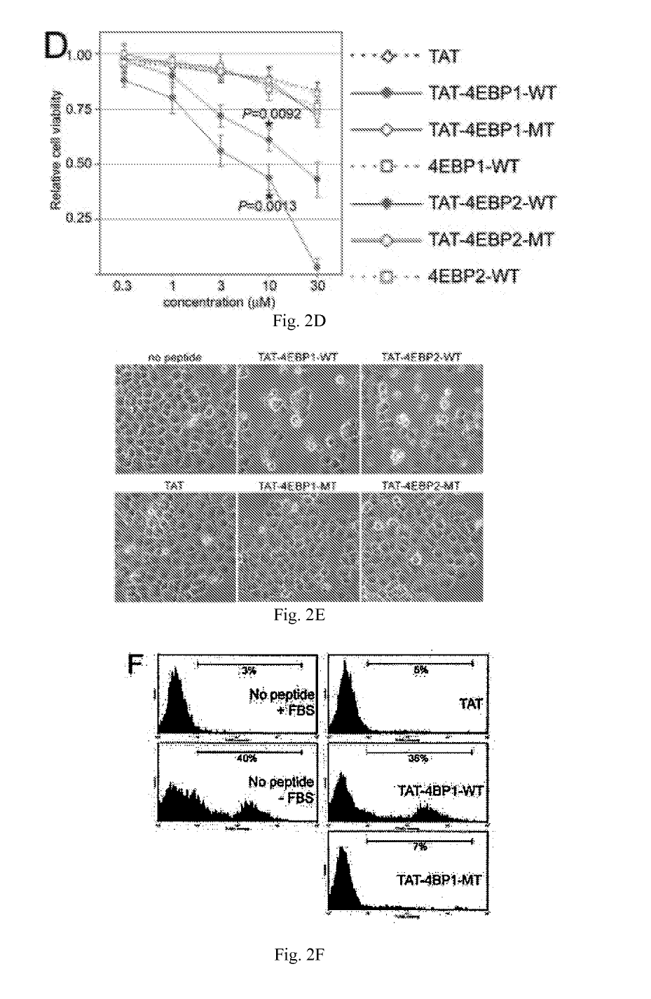

To determine efficacy of the peptides, a cell-based system was developed to assay changes in cap-dependent translation. This system is based on a bicistronic cassette in which one reporter, R-Luc, is synthesized in a cap-dependent manner, whereas the other, F-Luc, is synthesized under IRES-mediated control (Collier et al., 1998). Similar cassettes have been used for assaying translation in vitro (Moerke et al., 2007; Bordeleau et al., 2005). A cell line derived from the ovarian cancer cell line ES-2 was generated that stably expresses the bicistronic cassette (ES-2-DualLuc) (FIG. 2A). The robustness of the model system was determined using rapamycin, which inhibits cap-dependent translation by inhibiting mTOR from phosphorylating the 4EBPs (Beretta et al., 1996). Rapamycin inhibited cap-dependent translation (R-Luc read-out) in a dose-dependent manner, but did not inhibit cap-independent translation (F-Luc readout) (FIG. 2B). In contrast, the elongation inhibitor cycloheximide inhibited both cap-dependent and independent translation (FIG. 2B).

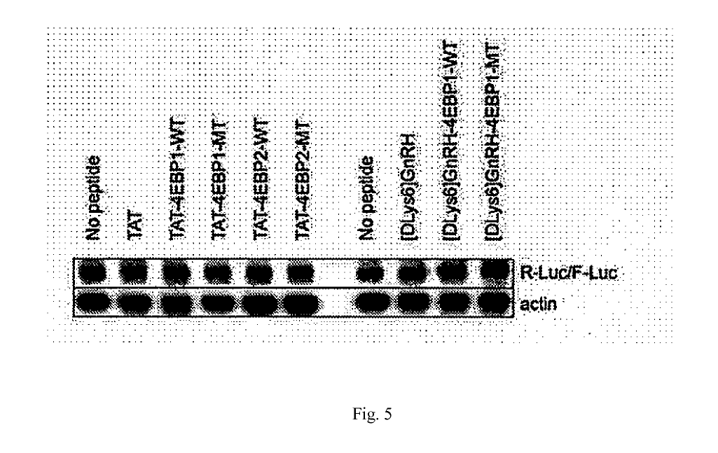

In ES-2-DualLuc cells, TAT-4EBP1-WT peptide inhibited cap-dependent translation but not cap-independent translation at concentrations ranging from 0.3 to 10 micromoles per liter (FIG. 2C). TAT-4EBP2-WT also inhibited cap-dependent translation, but was slightly less effective than TAT-4EBP1-WT. Under these conditions, peptide treatment did not alter the level of reporter transcript (FIG. 5). Cap-dependent translation was not inhibited by mutant peptides or by TAT alone at the same range of concentrations (FIG. 2C). At the highest concentration used (30 micromoles per liter), some inhibition of cap-independent translation by wild-type and mutant peptides was observed (FIG. 2C).

Treatment of OVCAR-3 cells with TAT-4EBP1-WT and TAT-4EBP2-WT peptides decreased cell viability in a dose-dependent manner, with TAT-4EBP1-WT demonstrating a stronger effect (FIG. 2D). In contrast, cell viability was not significantly affected by mutant peptides or by TAT at concentrations ranging from 0.3 to 10 micromoles per liter. Cell death was induced by wild-type TAT-4EBP peptides but not by mutant peptides (FIGS. 2E,F). Wild-type 4EBP peptides that lacked TAT did not affect cell viability (FIG. 2D), consistent with the inability of these peptides to internalize in cells (FIG. 1E).

### 4EBP Peptides Fused to a GnRH Agonist are Taken up by GnRH-RI-Expressing Cells

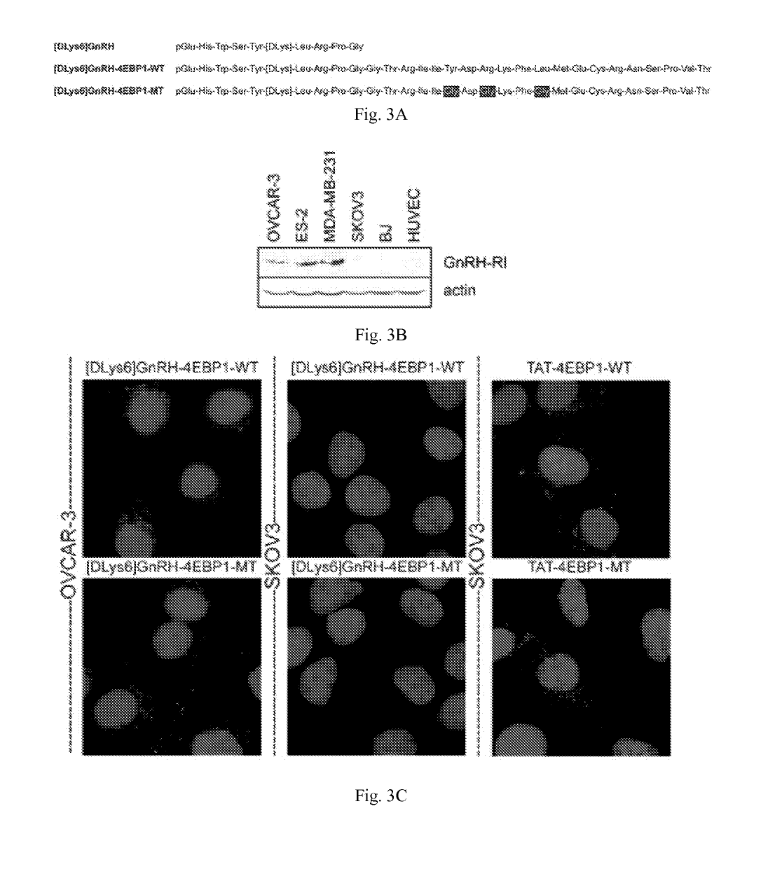

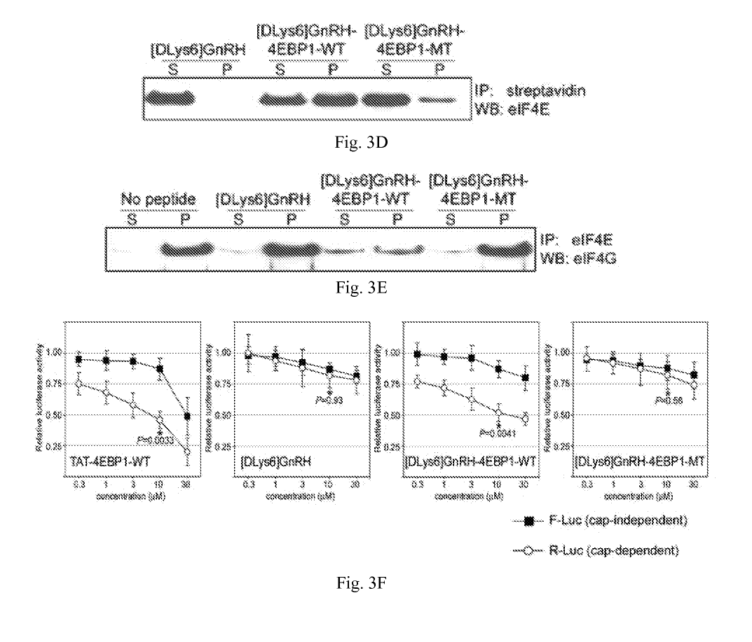

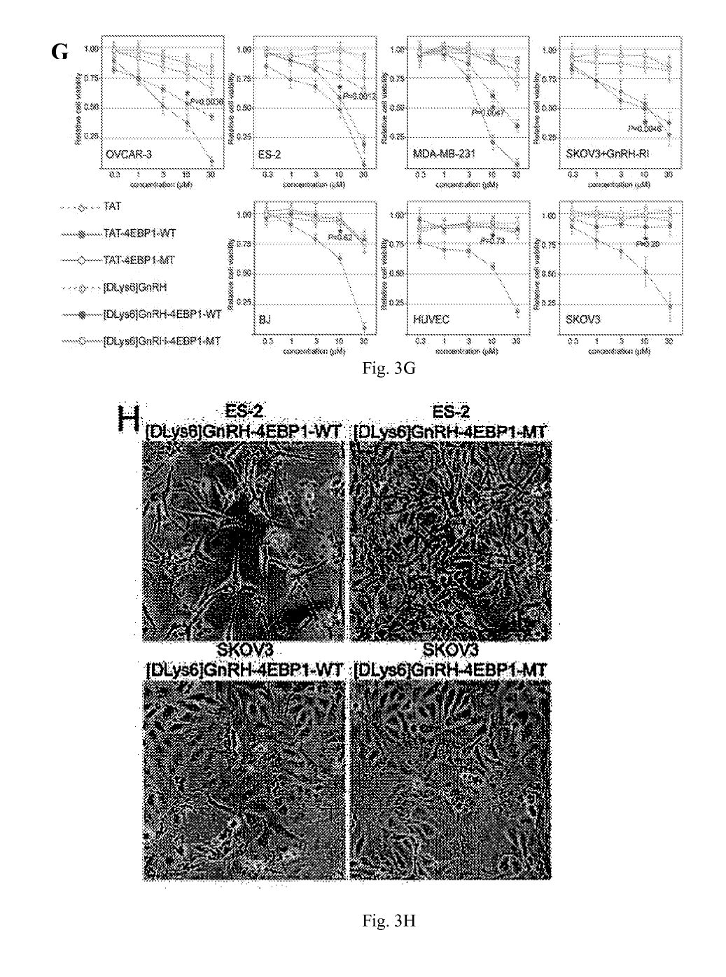

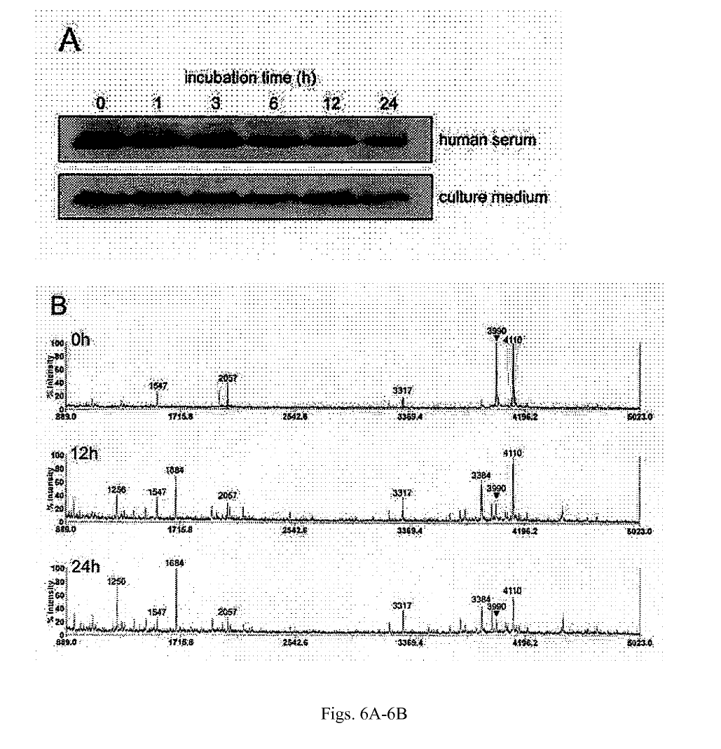

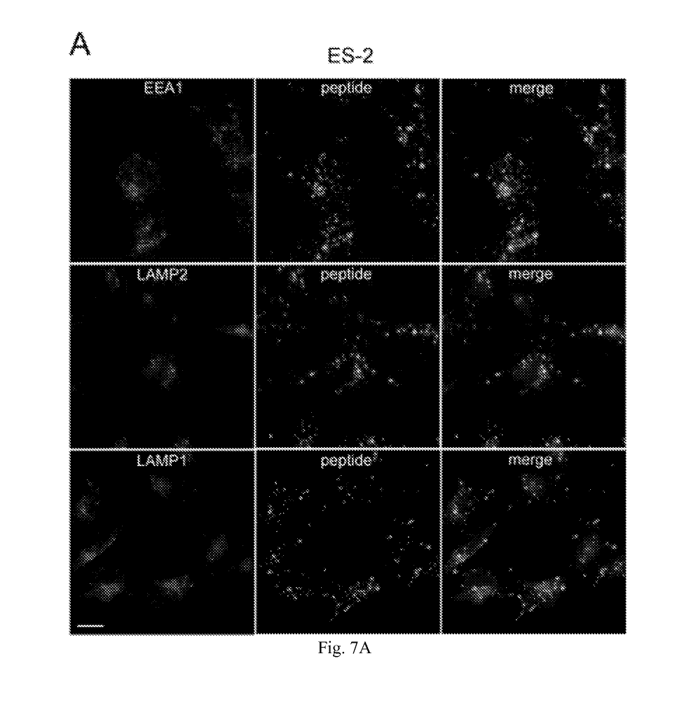

Because TAT does not facilitate peptide uptake in a cell type-specific manner, a wild-type 4BP1 peptide fused to the GnRH agonist [DLys6]GnRH (FIG. 3A) was generated. The 4EBP1 peptide was focused on because of its ability to bind eIF4E and prevent eIF4E from binding eIF4G was more effective than 4EBP2 peptide (FIGS. 1B,C). [DLys6]GnRH has been evaluated as a GnRH-RI-targeting moiety and, like almost all other clinically used GnRH agonists, contains a D-amino acid substitution at position 6 to promote stability (Millar et al., 2004; Miyazaki et al., 1997). Intact [DLys6]GnRH-4EBP1-WT fusion peptide was detected by western blot analysis following incubation at 37° C. in human serum and in culture medium containing FBS for up to 24 hours (FIG. 6A). The half-life of the peptide in serum was approximately 5 to 6 hours. The presence of intact peptide and its degradation following incubation in serum was confirmed by MALDI TOF mass spectrometry (FIG. 6B).

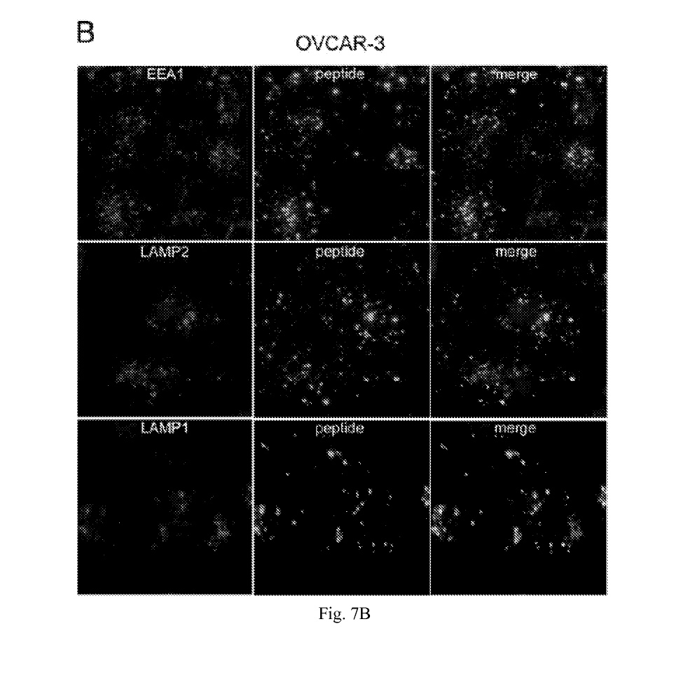

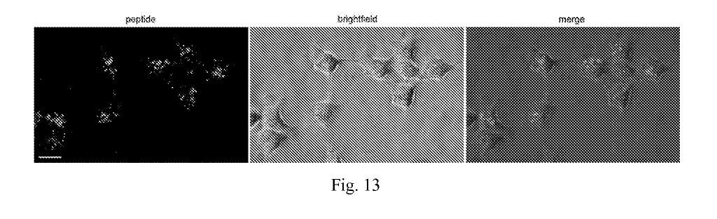

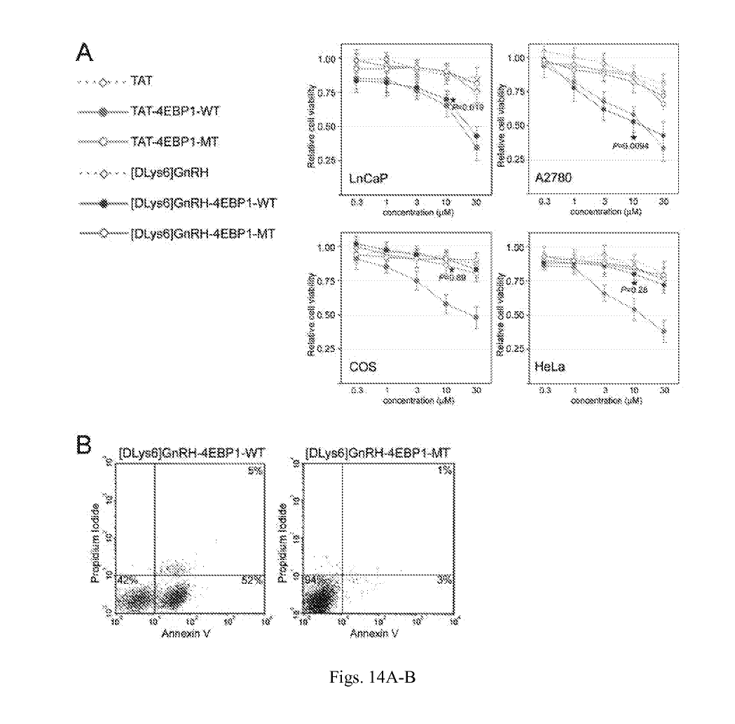

GnRH-RI was detected in OVCAR-3 and ES-2 cells and the breast cancer line MDA-MB-231, but not in the ovarian cancer cell line SKOV3, fibroblasts (BJ) and endothelial cells (HUVEC) (FIG. 3B). 4EBP1 peptide fused to [DLys6]GnRH was taken up by OVCAR-3 and ES-2 cells (FIG. 3C; FIGS. 7A,B). The possibility that intracellular localization of the peptide is an artifact of cell fixation was eliminated by visualizing FAM-labeled fusion peptide in live ES-2 cells that had been extensively washed following incubation with peptide and not subjected to fixation (FIG. 13). This data demonstrates that the [DLys6]GnRH-4EBP1-WT peptide is able to be taken up by live tumor cells. 4EBP1 peptide fused to TAT internalized in SKOV3 cells, but not peptide fused to [DLys6]GnRH (FIG. 3C).

[DLys6]GnRH-4EBP1-WT Peptide Binds eIF4E and Inhibits Cap-Dependent Translation in GnRHRI-Positive Cells

[DLys6]GnRH-4EBP1-WT peptide bound to eIF4E, whereas the GnRH alone did not bind (FIG. 3D). Only weak binding was observed with a fusion peptide [DLys6]GnRH-4EBP1-MT that contains mutations of the three critical binding residues of 4EBP1 (FIGS. 3A,D). [DLys6]GnRH-4EBP1-WT prevented eIF4E from binding eIF4G, whereas this binding was not inhibited by [DLys6]GnRH-4EBP1-MT or by [DLys6]GnRH alone (FIG. 3E). Because ES-2 cells express GnRH-RI (FIG. 3B), the ES-2-DualLuc reporter line was used to determine the effect of [DLys6]GnRH-4EBP1 peptides on translation. [DLys6]GnRH-4EBP1-WT inhibited cap-dependent translation without significantly inhibiting cap-independent translation, and was almost as effective as 4EBP1 peptide fused to TAT (FIG. 3F). In contrast, cap-dependent translation was not inhibited by mutant peptide or by [DLys6]GnRH (FIG. 3F).

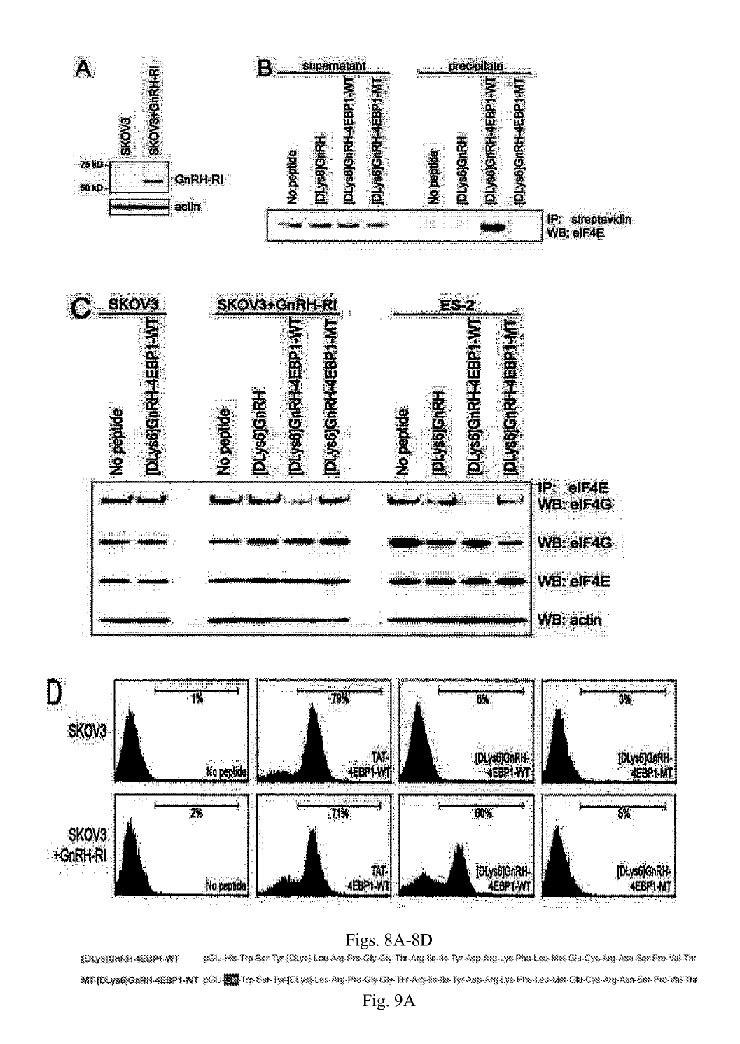

Although [DLys6]GnRH-4EBP1-WT inhibited cap-dependent translation in cells, it was important to determine that the peptide binds and inactivates its target within cells that express GnRH-RI. An SKOV3 cell line was generated that stably expresses GnRH-RI (FIG. 8A). Cells of this line (SKOV3+GnRH-RI) were cultured with peptides, and immunoprecipitation experiments performed using lysates of treated cells. Binding of [DLys6]GnRH-4EBP1-WT to eIF4E was detected, whereas neither mutant peptide nor [DLys6]GnRH alone bound eIF4E in cells treated with these peptides (FIG. 8B). Binding of eIF4E to eIF4G was substantially reduced in SKOV3+GnRH-RI cells treated with [DLys6]GnRH4EBP1-WT (FIG. 8C). The reduced binding of eIF4E to eIF4G was not due to any decrease in expression of these factors (FIG. 8C). On the other hand, binding of eIF4E to eIF4G was not inhibited in cells that had been treated with mutant peptide or with [DLys6]GnRH alone (FIG. 8C). The same findings were observed using the GnRH-RI-positive line ES-2 (FIG. 8C). In contrast, binding of eIF4E to eIF4G was not inhibited in GnRH-RI-negative parental SKOV3 cells following treatment with wild-type peptide (FIG. 8C).

### [DLys6]GnRH-4EBP1-WT Peptide Inhibits Growth of GnRH-RI-Positive Tumor Cells

Growth of GnRH-RI-positive lines OVCAR-3 (ovarian cancer), ES-2 (ovarian cancer), MDA-MB-231 (breast cancer), LnCaP (prostate cancer) and A2780 (ovarian cancer) was inhibited by [DLys6]GnRH4EBP1-WT in a dose-dependent manner, but not by [DLys6]GnRH-4EBP1-MT or by [DLys6]GnRH (FIG. 3G; FIG. 14A). Cell blebbing and detachment were induced by wild-type peptide but not by mutant peptide, even at the highest concentration tested (30 micromoles per liter) (FIG. 3H). Annexin V-binding was detected in GnRH-RI-positive cells following treatment with wild-type peptide, but not with mutant peptide (FIG. 14B). In contrast, [DLys6]GnRH-4EBP1-WT failed to inhibit growth of fibroblasts (BJ) and endothelial cells (HUVEC), kidney cells (COS) and HeLa cervical cancer cells that do not express GnRH-RI (FIG. 3G; FIG. 14A). Parental SKOV3 cells were likewise resistant to [DLys6]GnRH-4EBP1-WT, whereas this peptide inhibited growth and induced cell death, as indicated by TUNEL-staining, in SKOV3+GnRH-RI cells (FIG. 3G; FIG. 8D). On the other hand, GnRH-RI-positive and -negative lines were equally sensitive to 4EBP1-WT peptide fused to TAT (FIG. 3G; FIG. 8D; FIG. 14A). These observations indicate that wild-type 4EBP1 peptide fused to [DLys6]GnRH inhibits growth and induces cell death only in GnRH-RI-positive cells, but not in cells that do not express GnRH-RI. The morphologic changes, Annexin V-binding and TUNEL-staining detected in these cells indicate cell death occurs, at least in part, by apoptosis. The data regarding the ability of the peptide to inhibit growth of prostate cancer cells is particularly important for its therapeutic application.

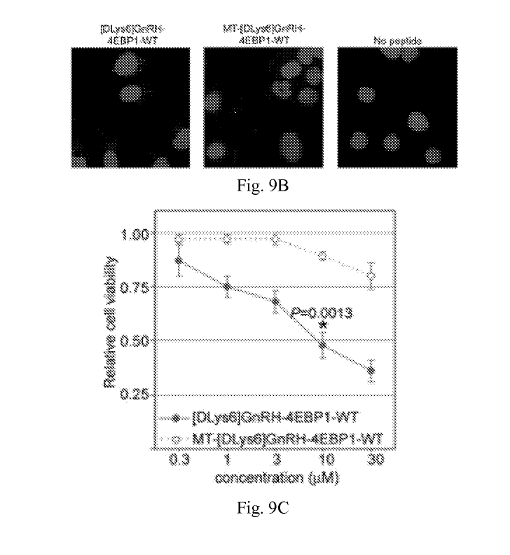

To confirm specificity of [DLys6]GnRH as a targeting moiety, a peptide was generated comprising 4EBP1-WT residues fused to a mutant agonist in which His-2 was substituted by Gln (FIG. 9A). His-2 of GnRH is essential for binding to GnRH-RI, and its substitution by Gln abolishes its activity (Millar et al., 2004; Yanaihara et al., 1973). In contrast to 4EBP1-WT fused to wild-type agonist, 4EBP1-WT fused to mutant agonist was not internalized in GnRH-RI-positive cells and did not inhibit cell growth (FIGS. 9B,C).

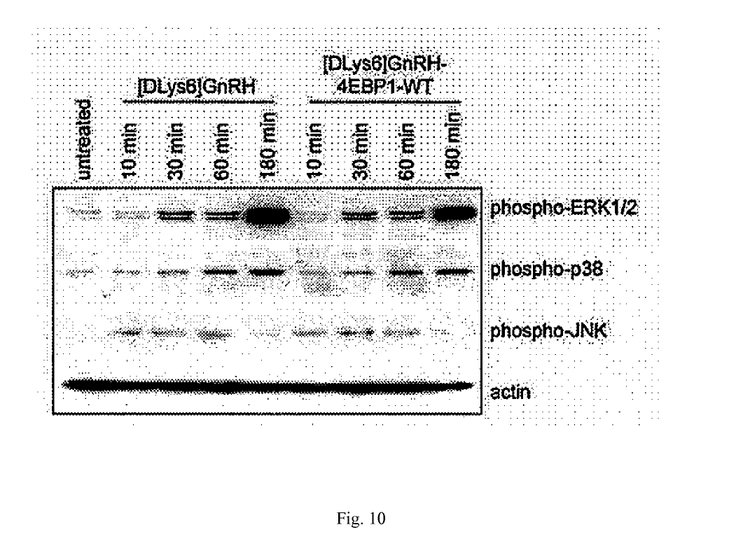

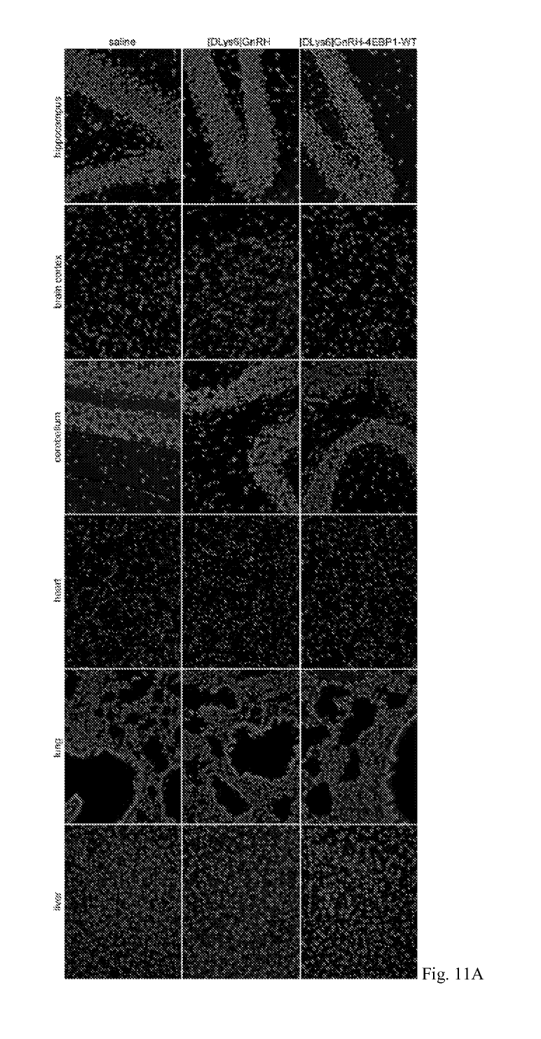

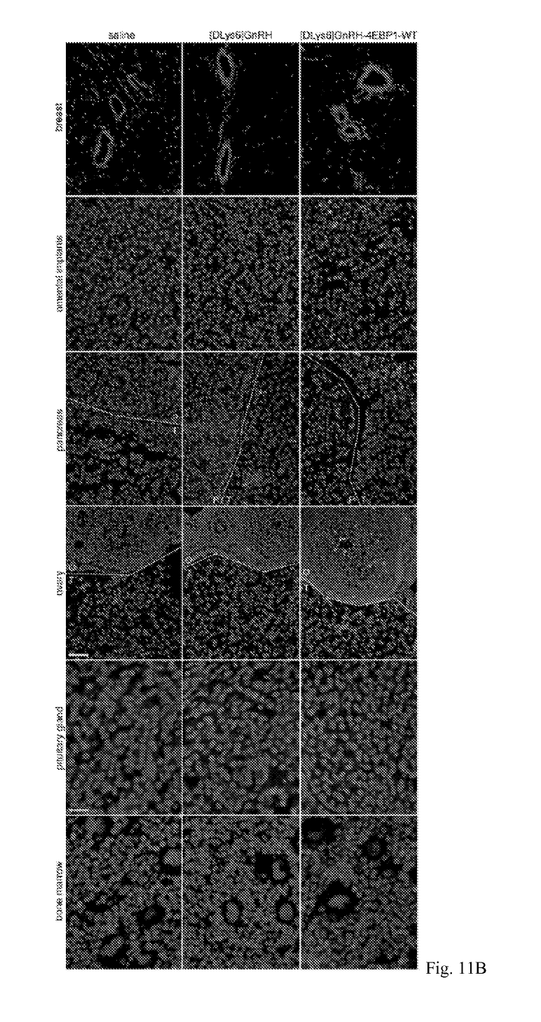

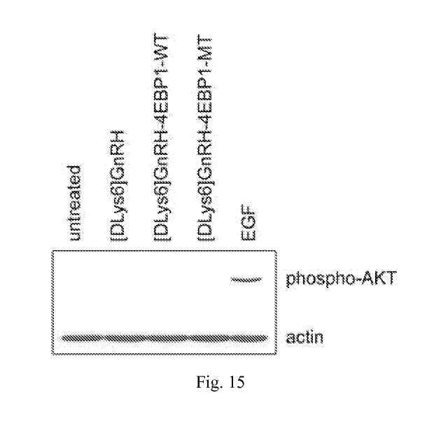

GnRH agonists generally exert only modest growth-inhibitory effects in ovarian cancer cells (Adelson and reece, 1993; Peterson et al., 1994). Indeed, [DLys6]GnRH alone did not significantly inhibit cell growth (FIG. 3G). GnRH-RI is a G protein-coupled receptor that activates MAPK cascades (Millar et al., 2004; Harrison et al., 2004). No significant difference was observed in activation of ERK1/2, p38 and JNK in ES-2 cells treated with [DLys6]GnRH-4EBP1-WT as compared to cells treated with [DLys6]GnRH alone (FIG. 10). This observation eliminates the possibility that the fusion peptide inhibits growth at least in part by altering GnRH agonist-mediated signaling in tumor cells. In addition, the fusion peptide did not induce AKT activation (FIG. 15). By providing evidence that treatment of cells with [DLys6]GnRH-4EBP1-WT peptide does not activate AKT, this data is important because feedback activation of AKT is a negative aspect of rapamycin, a drug that is commonly used to inhibit eIF4E activity.

These findings that [DLys6]GnRH-4EBP1-WT binds eIF4E, prevents eIF4E from binding eIF4G and inhibits cap-dependent translation in GnRH-RI-expressing cells strongly imply that an appreciable proportion of peptide enters the cytoplasm. Immunofluorescent staining revealed that peptides are mostly excluded from the nucleus (FIG. 3C). GnRH agonists are internalized in cells within 1 hour at 37° C. (28). At 1 hour after treatment of ES-2 cells with [DLys6]GnRH-4EBP1-WT at 37° C., some peptide was detected in early endosomes (visualized by staining of EEA1) (FIG. 7A). A small proportion of peptide was detected in late endosomes and lysosomes (visualized by staining of LAMP1 and LAMP2) at 1 hour (FIG. 7A), but this proportion did not increase with extended treatment (6 hours). Similar observations were made in OVCAR-3 cells (FIG. 7B).

### Anti-Tumor Effect of [DLys6]GnRH-4EBP1-WT Peptide in an EOC Xenograft Model

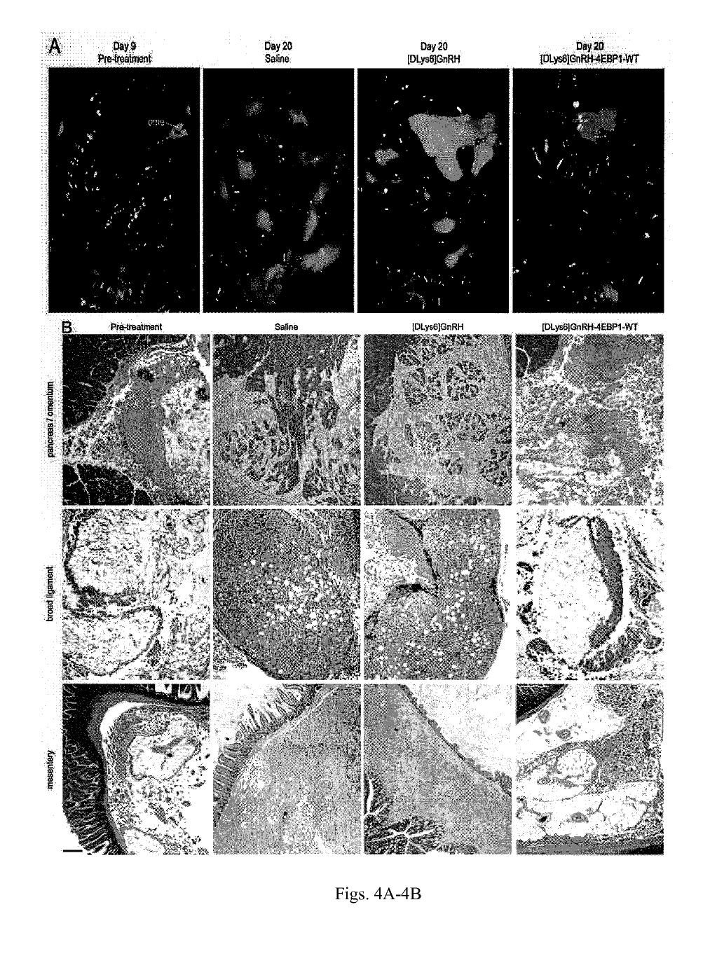

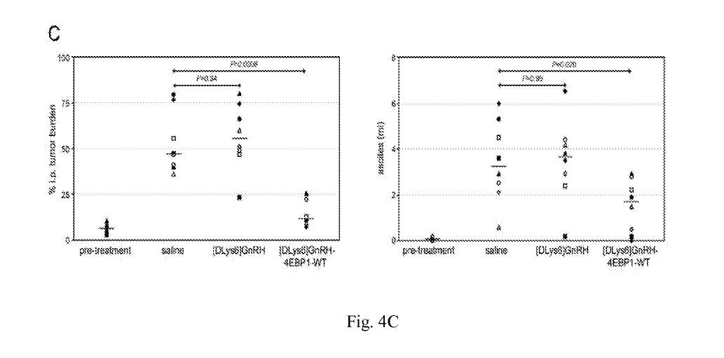

To determine the efficacy of [DLys6]GnRH-4EBP1-WT peptide in vivo, a mouse i.p. xenograft model was used that was established from an ES-2 cell line stably expressing GFP (Hara et al., 2007). This model mimics the typical behavior of epithelial ovarian cancer, including i.p. dissemination and ascites formation. This example comprised three treatment groups of mice i) saline, ii) [DLys6]GnRH agonist, and iii) [DLys6]GnRH-4EBP1-WT, where sample size of each group was n=11. The first dose of peptide or saline was administered i.p. to mice at Day 9 after tumor cell inoculation. At Day 9, no ascites had yet formed but tumors had established on the omentum, broad ligament and mesentery which are common sites of ovarian cancer cell attachment (FIGS. 4A,B). A schedule in which peptide was administered every 2 days was chosen, based on the studies of peptide stability in serum (FIG. 6).

Based on studies of other agonist conjugates in xenograft models (19-21), a peptide dose of 3.0 nanomoles per gram body weight was used. Duration of the regimen was 11 days (i.e. 6 doses in total), and mice were sacrificed on Day 20. At Day 20, saline-treated mice had developed ascites and extensive tumors throughout the abdominal cavity (FIGS. 4A,B,C). Table I summarizes treatment groups in the example, and Table II focuses on drop-out mice, or those mice which were euthanized as a result of ascites accumulation before the study completed.

TABLE I
Treatment Groups Profile

| | | | [DLys6]GnRH- |
|---|---|---|---|
| | Saline | [DLys6]GnRH | 4EBP1-WT |
| Total number of mice | 11 | 11 | 11 |
| per group | | | |
| Mice evaluated^{a} | 8 | 8 | 8 |
| Deaths^{b} | 1 | 2 | 1 |
| Drop-outs^{c} | 2 | 1 | 2 |

^{a}Completed the 20 day regimen.
^{b}Died before completing treatment regimen: saline group (n = 1, day 12); [DLys6]GnRH group (n = 2, day 17); and [DLys6]GnRH-4EBP1-WT group (n = 1, day 19)
^{c}Euthanized prior to completion of treatment regimen due to morbidity related to ascites accumulation.

Of the original 11 animals in each treatment group, 8 animals in each group completed the regimen and were evaluated. I.p. tumor burden, defined as percent of the abdominal cavity, was similar in mice treated with [DLys6]GnRH agonist alone (mean=56.4%, 95% confidence interval (CI)=41.5% to 71.3%) and in mice treated with saline (mean=52.8%, 95% CI=38.9% to 66.7%, P=0.34). In contrast, i.p. tumor burden was substantially smaller in mice treated with [DLys6]GnRH-4EBP1-WT peptide (mean=13.7%, 95% CI=8.4% to 19.1%, P=0.0008) (FIG. 4C). As compared with ascites volumes in saline-treated mice (mean=3.44 ml, 95% CI=1.95 ml to 4.93 ml), ascites volumes in mice treated with fusion peptide were also reduced (mean=1.44 ml, 95% CI=0.43 ml to 2.46 ml, P=0.020) (FIG. 4C).

TABLE II
Features of the Drop-out Mice ^{a}

| | | | [DLys6]GnRH- |
|---|---|---|---|
| | Saline | [DLys6]GnRH | 4EBP1-WT |
| Mice euthanized | n = 1, day 17 | n = 1, day 16 | n = 1, day 15 |
| | n = 1, day 19 | | n = 1, day 17 |
| Percent i.p. | 68.8% | 40.9% | 47.1% |
| tumor burden | 69.8% | | 27.8% |
| Ascites volume | 6.2 mL | 8.9 mL | 10.0 mL |
| | 5.5 mL | | 6.2 mL |

^{a} Euthanized prior to completion of treatment regimen due to morbidity related to ascites accumulation.

During the course of treatment, mice in control and treated groups were observed to maintain appetite. Neither alterations in urine and stools nor dermal changes were noted. Impairment of corneal and pedal reflexes was not observed, but mobility was reduced in mice that developed extensive ascites. No gross histologic changes were seen in normal tissues of peptide-treated mice. No TUNEL-staining was detected in breast and pituitary tissues that express GnRH-RI, but some staining was observed in ovaries of mice treated with [DLys6]GnRH-4EBP1-WT (FIG. 11). TUNEL-positive cells were not detected in other normal tissues of these mice, such as brain, heart, lung and bone marrow. Cells of the liver and pancreas were also TUNEL-negative, whereas TUNEL-positive cells were detected in adjacent tumors (FIG. 11).

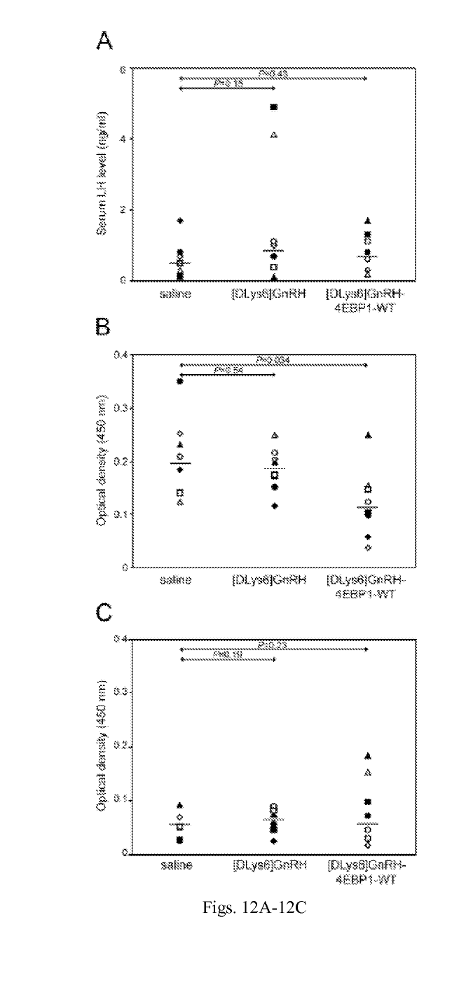

To further assess for pituitary damage, levels of luteinizing hormone (LH) were assayed in serum collected at the end of the regimen. Serum LH levels of mice treated with [DLys6]GnRH4EBP1-WT were not significantly different from LH levels of saline-treated mice, whereas elevated LH levels were detected in 2 of 8 mice treated with [DLys6]GnRH alone (FIG. 12A). The immunogenicity of [DLys6]GnRH-4EBP1-WT was also determined by assaying mouse serum antibodies for reactivity to the peptide. As a positive control, antibody reactivity to cellular proteins of ES-2 cells was assayed. Whereas antibody reactivity to tumor cell proteins was detected in all groups of mice, antibody reactivity to [DLys6]GnRH-4EBP1-WT that was at least two standard deviations above the mean background level in saline-treated mice was detected in 2 of 8 peptide-treated mice (FIGS. 12B,C).

### Discussion

Inhibiting translation initiation is a candidate approach for cancer therapy, but presents significant challenges. Rapamycin inhibits 4EBP phosphorylation (Beretta et al., 1996), but its growth-inhibitory effect is attenuated in part by its ability to induce feedback activation of AKT signaling (Sun et al., 2005). Increasing attention has focused on other agents that inhibit eIF4E and other components of the translation initiation complex Tumstatin, a fragment of type IV collagen, interacts with alphaV.beta3.integrin and prevents dissociation of eIF4E from 4EBP1 (Maeshima et al., 2002). Other agents include 4EGI-1, a small molecule inhibitor that prevents eIF4E from binding to eIF4G (3), RNA aptamers that bind eIF4G (31), and the marine natural products pateamine and hippuristanol that target the RNA helicase eIF4A (Bordeleau et al., 2005; Bordeleau et al., 2006). However, specificity of these agents for tumor cells and lack of cytotoxicity in normal cells has not been demonstrated in animal models. It has been reported that eIF4E antisense oligonucleotides inhibit tumor xenograft growth, but also substantially inhibit eIF4E levels in the liver (Graff et al., 2007).

Based on X-ray crystal structural studies of the interaction of 4EBPs with eIF4E (Tomoo et al., 2005; Marcotrigiano et al., 1999), 4EBP peptides that bind eIF4E and prevent eIF4E from binding to eIF4G were generated. These peptides were fused to TAT in initial proof-of-concept studies to facilitate cellular uptake. Cap-dependent translation and cell growth were inhibited by wild-type TAT-4EBP peptides, but not by fusion peptides containing mutations that impaired their ability to bind eIF4E and to prevent eIF4E from binding eIF4G. These findings differ from a study in which eIF4E-binding peptides were fused to the non cell-specific transporter penetratin and evaluated in the MRCS lung cells in vitro (Herbert et al., 2000). In the earlier study, the peptides only induced cell death under serum-starved conditions, and their ability to inhibit the translational function of eIF4E was not determined. One significant difference is that peptides described in this invention comprise 4EBP residues 49-68, whereas peptides in the earlier study contained residues 51-62 (Herbert et al., 2000). Trp-73 of eIF4E is important for 4EBP binding, and interacts with Arg-63 of 4EBP1 and 4EBP2 (Tomoo et al., 2005; Marcotrigiano et al., 1999). The additional 4EBP residues in peptides described in this invention, including Arg-63, might strengthen their ability to bind eIF4E and prevent eIF4G from binding eIF4E.

In this study, inhibition of cap-dependent translation was detected as early as 6 hours in the cell-based assay system when no significant changes in cell viability had yet occurred. This implies that the peptides induce cell death by inhibiting the translational function of eIF4E. It should be noted that cap-independent translation was inhibited by TAT-4EBP peptides at the highest concentration used (30 micromoles per liter). Therefore, the possibility cannot be excluded that 4EBP peptides might induce cell death in part by inhibiting a pathway independent of eIF4E's role in translation or induce other cytotoxic effects.

It is intriguing to note that the small molecule inhibitor 4EGI-1 not only blocks eIF4E from binding eIF4G but also enhances binding of 4EBP1 to eIF4E (Moerke et al., 2007). Displacement of eIF4G from eIF4E by 4EGI-1 was speculated to free the binding site for 4EBP1 by removing steric obstruction (Moerke et al., 2007). Because the peptides described in this invention contain 4EBP residues, it is not surprising that these peptides do not promote binding of 4EBP1 to eIF4E (unpublished data). It is also interesting to note a recent report that elevated levels of 4EBP1 and eIF4G promote a hypoxia-activated switch from cap-dependent to IRES-dependent synthesis of VEGF and HIF-1 alpha (Braunstein et al., 2007). This raises the possibility that a 4EBP1-mimicking peptide might promote tumor growth. However, this was not found to be the case for the 4EBP1 peptide fused to [DLys6]GnRH. Mice that completed treatment with [DLys6]GnRH-4EBP-WT fusion peptide showed a 50% reduction in tumor burden and ascites as compared to mice treated with saline or the GnRH agonist alone. Of the 11 mice treated with [DLys6]GnRH-4EBP-WT, one died and two were terminated prior to completing the regimen. However, the death and drop-out rates of the peptide-treated group were not higher than rates in control groups. Furthermore, the tumor burden of peptide-treated drop-out mice was not higher than that of drop-outs of control groups. Moreover, HIF-1 alpha expression in tumors was not higher in peptide-treated drop-out mice, as compared to drop-outs of control groups.

An important aspect of this work is the fusion of 4EBP peptide to a GnRH agonist as a targeting moiety. Cell-penetrating peptides such as penetratin and TAT do not facilitate peptide uptake in a cell type-specific manner. Indeed, it was observed that 4EBP peptide fused to TAT significantly inhibited growth of endothelial cells and fibroblasts. The rationale for using a GnRH agonist as a targeting moiety was based on several considerations. One significant consideration is the high prevalence of GnRH-RI overexpression in ovarian cancer and other endocrine cancers, and its relatively restricted tissue distribution (Harrison et al, 2004; Imai et al., 1994). GnRH-RI is expressed in the ovary, uterus, breast, prostate and pituitary (Harrison et al, 2004). An important concern with using a GnRH agonist is the potential for pituitary toxicity. In this example, cell death was not detected in pituitary tissues of mice treated with [DLys6]GnRH-4EBP1-WT peptide. In addition, LH levels in these mice were not significantly altered, indicating that pituitary function was not impaired. Alterations in reproductive capability could not be assayed due to rapid growth of tumors. However, increased TUNEL-staining was observed in ovaries of mice treated with [DLys6]GnRH-4EBP1-WT peptide.

A second factor in choosing a GnRH agonist as a targeting moiety is its small size. Fusion of wild-type 4EBP1 peptide to [DLys6]GnRH did not interfere with its ability to bind eIF4E and to prevent eIF4E from binding eIF4G. A third factor is the capability of GnRH agonists to facilitate cellular uptake. Folic acid has been studied as a targeting moiety, but one disadvantage is low efficiency of folate receptor internalization (Paulos et al., 2004). On the other hand, GnRH agonists are efficiently internalized by receptor-mediated endocytosis and, shortly thereafter, dissociate from the receptor (Cornea et al., 1999). GnRH agonists have been used as delivery vehicles for peptides that inhibit cytoplasmic proteins. These include Pseudomonas exotoxin that inactivates elongation factor-2 through ADP ribosylation (Nechushtan et al., 1997), and pokeweed antiviral protein, an RNA N-glycosidase that inactivates ribosomes by inducing conformational change (Qi et al., 2004). The mechanism by which these peptides exit endosomes is unclear. In this example, an appreciable proportion of internalized [DLys6]GnRH-4EBP-WT peptide did not localize to late endosomes and lysosomes. Moreover, binding of the peptide to eIF4E was strongly detected in GnRH-RI-expressing tumor cells that had been treated with this peptide, and binding of eIF4E to eIF4G was inhibited in these cells. The possibility that wild-type peptide might inhibit growth in part by mechanisms unrelated to inhibiting eIF4E activity cannot be totally excluded, but appears neither to involve alteration of GnRH-RI signaling in tumor cells nor a suppression of LH levels.

Seventy percent of patients diagnosed with epithelial ovarian cancer present with disseminated disease. For these patients, the 5-year survival rate is only 30% and most will eventually die of the disease. For many years, tumour-debulking surgery and taxane-platinum combination therapy have remained the standard-of-care. Relapse is frequent and the high initial response rate does not translate to a high cure rate. New-generation agents that target molecular focal points that drive tumor growth and metastasis are therefore essential. The proof-of-concept suggested that this invention may be used in ovarian cancer and also other endocrine tumors that express GnRH-RI.

### Abbreviations

The following abbreviations are used herein: eIF4E, eukaryotic translation initiation factor 4E; eIF4G, eukaryotic translation initiation factor 4G; Ab, antibody; Abs, antibodies; F-Luc, firefly luciferase; GFP, green fluorescent protein; GnRH, gonadotropin-releasing hormone; GnRH-RI, gonadotropin-releasing hormone receptor I; HUVEC, human vascular endothelial cells; IRES, internal ribosome entry site; LH, luteinizing hormone; R-Luc, Renilla luciferase; 4EBP, eIF4E-binding protein, 5′-UTR, 5′-untranslated region.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

### Patent Documents:

U.S. Pat. No. 7,141,541
U.S. Pat. No. 6,410,715
U.S. Pat. No. 6,111,077
U.S. Pat. No. 5,874,231
International Patent PCT/US2006002093

### Manuscript References:

Adelson and Reece, Clin. Obstet. Gynecol., 36:690-700, 1993.
Beretta et al., EMBO. J., 15:658-664, 1996.
Berg et al. Clin Cancer Res., 11(23):8476-8485, 2005.
Biecker et al. J. Pharmacol. Exp. Ther., 313(3):952-961, 2005.
Bordeleau et al., Nat. Chem. Biol., 2:213-220, 2006.
Bordeleau et al., Proc. Natl. Acad Sci. USA, 102:10460-10465, 2005.
Braunstein et al., Mol. Cell, 28:501-512, 2007.
Caruso et al. Mol Pharmacol., 65(4):1016-1028, 2004.
Chandna et al., Mol. Pharm., 4:668-678, 2007.
Collier et al., J. Gen. Virol., 79:2359-2366, 1998.
Cornea et al., Endocrinology, 140:4272-4280, 1999.
De Benedetti and Graff, Oncogene, 23:3189-3199, 2004.
Dharap et al., Proc. Natl. Acad. Sci. USA, 102:12962-12967, 2005.
Fletcher et al Biochemistry, 37:9-15, 1998.
Gao et al. J Hypertens., 24(8):1663-1670, 2006.
Graff et al., J. Clin. Invest., 117:2638-2648, 2007.
Hara et al., Am. J. Pathol., 170:1595-1606, 2007.
Harrison et al., Endocr. Relat. Cancer, 11:725-748, 2004.
Herbert et al., Curr. Biol., 10:793-796, 2000.
Houbenweyl, In: Methods of Organic Chemistry, Wansch (Ed.), Vol. 15:I and II, Thieme, Stuttgart, 1987.
Imai et al., Cancer, 74:2555-2561, 1994.
Kevil et al., Int. J. Cancer, 65:785-790, 1996.
Lavignac et al. Int. J. Pharm., 300(1-2):102-12, 2005.
Lazaris-Karatzas et al., Nature, 345:544-547, 1990.
Maeshima et al., Science, 295:140-143, 2002.
Mamane et al., Oncogene, 23:3172-3179, 2004.
Mamane et al., PLoS ONE, 2:e242, 2007.
Marcotrigiano et al., Mol. Cell, 3:707-716, 1999.
Merrifield, J. Am. Chem. Assoc., 85:2149 2154, 1964.
Michiue et al., J. Biol. Chem., 280:8285-8289, 2005.
Millar et al., Endocr. Rev., 25:235-275, 2004.
Miyakawa et al., RNA, 12:1825-1834, 2006.
Miyazaki et al., J. Natl. Cancer Inst., 89:1803-1809, 1997.
Moerke et al., Cell, 128:257-267, 2007.
Nechushtan et al., J. Biol. Chem., 272:11597-11603, 1997.
Paulos et al., Mol. Pharmacol., 66:1406-1414, 2004.
Peterson et al Gynecol. Oncol., 52:26-30, 1994.
Proud, Biochem. J., 403:217-234, 2007.
Qi et al., Cancer Res., 64:2090-2095, 2004.
Raghuraman et al. Biophys J., 92(4):1271-1283, 2007.
Remington's Pharmaceutical Sciences, 21^{st} Ed. Lippincott Williams & Wilkins, 2005.
Rousseau et al., Proc. Natl. Acad. Sci. USA, 93:1065-1070, 1996.
Ruggero et al., Nat. Med., 10:484-486, 2004.
Shiah et al. Clin Cancer Res., 6:1008-1015, 2000.
Shiraishi et al. FEBS Lett., 580(5):1451-1456, 2006.
Sun et al., Cancer Res., 65:7052-7058, 2005.
Tomoo et al., Biochim. Biophys. Acta., 1753:191-208, 2005.
Yanaihara et al., Biochem. Biophys. Res. Comm., 51:165-173, 1973.

1. A chimeric peptide construct comprising an eIF4E binding domain and one or more domains selected from the group consisting of a cell targeting domain, a cell-penetrating domain and a cytoplasmic delivery domain, wherein the eIF4E binding domain inhibits protein synthesis.

2. The construct of claim 1, wherein the eIF4E binding domain comprises a sequence selected from the group consisting of SEQ ID NO:2, SEQ ID NO:3, SEQ ID NO:4, and SEQ ID NO:5.

3. The construct of claim 1, wherein the cell targeting domain is a gonadotropin-releasing hormone receptor binding domain, and wherein said chimeric peptide construct binds preferentially to cells having a gonadotropin receptor.

4. The construct of claim 1, wherein the cell targeting domain comprises SEQ ID NO:1.

5. The construct of claim 1, wherein the cytoplasmic delivery domain is selected from the group consisting of an influenza virus hemagglutinin-2 sequence, a photosensitizer, and a melittin-derived peptide.

6. The construct of claim 1, wherein the cell-penetrating domain is a peptide derived from the transactivating (TAT) protein of the human immunodeficiency virus (HIV).

7. The construct of claim 1, wherein said construct comprises a cell targeting domain and an eIF4E binding domain.

8. The construct of claim 7, wherein the cell targeting domain is a gonadotropin-releasing hormone receptor binding domain, and wherein said chimeric peptide construct binds preferentially to cells having a gonadotropin receptor.

9. The construct of claim 7, wherein said construct comprises SEQ ID NO:6 or SEQ ID NO:7.

10. The construct of claim 7, wherein said construct further comprises a cytoplasmic delivery domain.

11. The construct of claim 10, wherein the cytoplasmic delivery domain is selected from the group consisting of an influenza virus hemagglutinin-2 sequence, a photosensitizer, and a melittin-derived peptide.

12. The construct of claim 1, wherein said construct comprises a cell-penetrating domain and an eIF4E binding domain.

13. The construct of claim 12, wherein the cell-penetrating domain is a peptide derived from the transactivating (TAT) protein of the human immunodeficiency virus (HIV).

14. The construct of claim 13, wherein the cell-penetrating domain comprises SEQ ID NO:8.

15. The construct of claim 12, wherein said construct further comprises a cytoplasmic delivery domain.

16. The construct of claim 15, wherein the cytoplasmic delivery domain is selected from the group consisting of an influenza virus hemagglutinin-2 sequence, a photosensitizer, and a melittin-derived peptide.

17. The construct of claim 12, wherein said construct further comprises a cell targeting domain.

18. The construct of claim 17, wherein the cell targeting domain is a gonadotropin-releasing hormone receptor binding domain, and wherein said chimeric peptide construct binds preferentially to cells having a gonadotropin receptor.

19. The construct of claim 18, wherein the cell targeting domain comprises SEQ ID NO:1.

20. The construct of claim 1, wherein said construct comprises a cell-penetrating domain, a cell targeting domain, a cytoplasmic delivery domain, and an eIF4E binding domain.

21. A method of treating a condition characterized by abnormal cell proliferation or abnormal cell survival in a subject, comprising:
administering to a subject in need of such treatment, a therapeutically effective amount of the chimeric peptide construct of claim 1.

22. The method of claim 21, wherein the condition characterized by abnormal cell proliferation or abnormal cell survival is an endocrine cancer.

23. The method of claim 22, wherein the endocrine cancer is selected from the group consisting of ovarian cancer, breast cancer, prostate cancer, endometrial cancer, cervical cancer, uterine cancer, and pituitary cancer.

24. The method of claim 23, wherein the endocrine cancer is ovarian cancer.

25. The method of claim 21, wherein the condition characterized by abnormal cell proliferation or abnormal cell survival is a cancer expressing a GnRH receptor.

26. The method of claim 25, wherein the cancer expressing a GnRH receptor is selected from the group comprising an intracranial tumor, a lymphoma, a melanoma, and a squamous cell carcinoma.

27. The method of claim 21, wherein the chimeric peptide construct is administered to the subject by intraperitoneal injection.

28. A pharmaceutical composition comprising one or more pharmaceutically acceptable excipients and a chimeric peptide construct of claim 1.

29. The composition of claim 28, wherein the chimeric peptide construct comprises SEQ ID NO:6 and SEQ ID NO:7.

30. A method of treating a condition associated with abnormal protein synthesis in a subject comprising:
administering to a subject in need of such treatment, a therapeutically effective amount of the chimeric peptide construct of claim 1.

31. A method of inhibiting protein synthesis in a GnRH receptor-bearing cell comprising:
contacting the cell with the chimeric peptide construct of claim 1 to inhibit protein synthesis.

32. A kit for treating a condition associated with abnormal cell proliferation or abnormal cell survival comprising a container, and a metered quantity of the pharmaceutical composition comprising the chimeric peptide construct of claim 1 disposed therein.

33. The kit of claim 32, further comprising an applicator.

34. The kit of claim 32, wherein the applicator is selected from the group consisting of a syringe, an intravenous infusion assembly, an intraperitoneal injection assembly, an applicator for topical administration, and an applicator for subcutaneous administration.
